(19) United States
(12) Patent Application Publication
(10) Pub. No.: US 2006/0257901 A1
(43) Pub. Date: Nov. 16, 2006

(54) Methods of diagnosing fetal trisomy 13 or a risk of fetal trisomy 13 during pregnancy

(75) Inventor: S. Ananth Karumanchi, Chestnut Hill, MA (US)

Correspondence Address: CLARK & ELBING LLP, 101 FEDERAL STREET, BOSTON, MA, 02110, US

(73) Assignee: Beth Israel Deaconess Medical Center, Boston, MA (US)

(21) Appl. No.: 11/389,638

(22) Filed: Mar. 24, 2006

Related U.S. Application Data
(60) Provisional application No. 60/664,756, filed on Mar. 24, 2005.

(51) Int. Cl. C12Q 1/68; G01N 33/567

(52) U.S. Cl. 435/6; 435/7.2

(57) ABSTRACT

Disclosed herein are methods for diagnosing fetal trisomy 13, or a risk of fetal trisomy 13, during pregnancy by detecting the levels of sFlt-1, VEGF, and PlGF in a subject.

### CROSS REFERENCE TO RELATED APPLICATIONS

This application claims the benefit of U.S. Provisional Application No. 60/664,756, filed on Mar. 24, 2005, herein incorporated by reference.

### STATEMENT AS TO FEDERALLY SPONSORED RESEARCH

This research was funded, in part, by NIH Grant R03 DK064255-02. The U.S. government has certain rights to the invention.

### FIELD OF THE INVENTION

In general, this invention relates to the detection of fetal trisomy 13 or a risk of having a fetus with trisomy 13 during pregnancy.

### BACKGROUND OF THE INVENTION

Pre-eclampsia is a syndrome of hypertension, edema, and proteinuria that affects 5 to 10% of pregnancies and results in substantial maternal and fetal morbidity and mortality. Pre-eclampsia accounts for at least 200,000 maternal deaths worldwide per year. The symptoms of pre-eclampsia typically appear after the 20^{th} week of pregnancy and are usually detected by routine monitoring of the woman's blood pressure and urine. However, these monitoring methods are ineffective for diagnosis of the syndrome at an early stage.

The proper development of the fetus and the placenta is mediated by several growth factors. One of these growth factors is vascular endothelial growth factor (VEGF). VEGF is an endothelial cell-specific mitogen, an angiogenic inducer, and a mediator of vascular permeability. VEGF has also been shown to be important for glomerular capillary repair. VEGF binds as a homodimer to one of two homologous membrane-spanning tyrosine kinase receptors, the fms-like tyrosine kinase (Flt-1) and the kinase domain receptor (KDR), which are differentially expressed in endothelial cells obtained from many different tissues. Flt-1, but not KDR, is highly expressed by trophoblast cells which contribute to placental formation. Placental growth factor (PlGF) is a VEGF family member that is also involved in placental development. PlGF is expressed by cytotrophoblasts and syncytiotrophoblasts and is capable of inducing proliferation, migration, and activation of endothelial cells. PlGF binds as a homodimer to the Flt-1 receptor, but not the KDR receptor. Both PlGF and VEGF contribute to the mitogenic activity and angiogenesis that are critical for the developing placenta.

Soluble Flt-1 receptor (sFlt-1) is a splice variant of the Flt-1 receptor which lacks the transmembrane and cytoplasmic domains. sFlt-1 binds to VEGF and PlGF with high affinity but does not stimulate mitogenesis of endothelial cells. sFlt-1 is believed to act as a “physiologic sink” to down-regulate VEGF signaling pathways. Regulation of sFlt-1 levels therefore works to modulate VEGF and PlGF and their respective signaling pathways, which is critical for maintaining appropriate proliferation, migration, and angiogenesis by trophoblast cells in the developing placenta.

We have previously discovered that sFlt-1 levels are elevated in blood serum samples taken from pre-eclamptic women. sFlt-1 binds to VEGF and PlGF with high affinity and blocks the mitogenic and angiogenic activity of these growth factors. Thus, sFlt-1, VEGF, and PlGF are useful diagnostic markers and therapeutic targets for pre-eclampsia. We have also previously discovered that free PlGF levels in the urine can be used as a diagnostic tool to detect pre-eclampsia or eclampsia, or a predisposition thereto.

Trisomy 13 is a chromosomal aberration that occurs in about 1 in 5,000 live births and accounts for 6 in 100 spontaneous abortions. Affected newborns have multiple severe malformations. Most fetuses having this anomaly are spontaneously aborted during the first two trimesters of pregnancy, and many prenatally diagnosed pregnancies are terminated. Because prenatal diagnosis usually occurs following amniocentesis, such terminations are relatively late in the pregnancy and are often traumatic. Half of the trisomy 13 newborns die within a month and only 5-10% survive beyond the first year. Second and third trimester trisomy 13 pregnancies are more prone to developing pre-eclampsia.

There is a need for methods of accurately diagnosing pregnant subjects having a fetus with trisomy 13, or at risk for having a fetus with trisomy 13, particularly early in pregnancy.

### SUMMARY OF THE INVENTION

We have discovered that serum levels of sFlt-1 are increased in women carrying trisomy 13 fetuses, possible due to the extra copy of the placental Flt-1 gene. We have also discovered that in women carrying trisomy 13 fetuses, there is a decrease in the level of PlGF and an increase in the level of the sFlt-1/PlGF ratio. The alteration in the circulating angiogenic state may be responsible for the increased risk of pre-eclampsia observed in these patients. We have discovered that the levels of sFlt-1, PlGF, and VEGF, as well as the ratio of sFlt-1/PlGF can be used as a diagnostic marker to screen for pregnant woman carrying or at risk for carrying a trisomy 13 fetus. The diagnostic methods and kits of the invention can be used alone or in combination with additional methods to confirm the genetic defect, including but not limited to ultrasound, amniocentesis, fluorescence in situ hybridization (FISH), and chronic villous sampling (CVS). The diagnostic methods of the invention can also be used as a preliminary screen to identify women who should undergo further testing for the fetal trisomy 13 defect.

Accordingly, in a first aspect, the invention features a method of diagnosing a pregnant subject as having, or at risk for having, a fetus with trisomy 13 that includes measuring the level of sFlt-1, VEGF, or PlGF polypeptide in a sample from the subject. This method can be used to determine absolute levels of sFlt-1, VEGF, or PlGF polypeptide that are below a threshold level and diagnose a pregnant subject as having, or at risk for having, a fetus with trisomy 13. For example, a serum level of sFlt-1 greater than 2 ng/ml bodily fluid (e.g., blood, serum, or urine) is a diagnostic indicator of fetal trisomy 13 or an elevated risk for having a fetus with trisomy 13. In another example, a serum value of free PlGF less than 50 pg/ml at 10-12 weeks, less than 100 pg/ml at 13-16 weeks, less than 200 pg/ml at 17-20 weeks, less than 300 pg/ml at 21-24 weeks, 25-28 weeks, 29-32 weeks, or 33-37 weeks, and less than 250 pg/ml at 37 to 41 weeks. This method can also be used to determine relative levels of sFlt-1, VEGF, or PlGF as compared to a reference sample where an alteration (e.g., increase or decrease of at least 10%, 20%, 30%, 40%, 50%, 60%, 70%, 80%, 90%, 95% or more) in the level of sFlt-1, VEGF, or PlGF as compared to a normal reference sample diagnoses a pregnant subject as having, or at risk for having, a fetus with trisomy 13. In this embodiment, an increase in the level of sFlt-1 or a decrease in the level of free VEGF or free PlGF polypeptide from the subject sample compared to a reference sample is a diagnostic indicator of fetal trisomy 13 or a subject at risk for having a fetus with trisomy 13.

In a related aspect, the invention provides a method of diagnosing a pregnant subject as having, or at risk for having, a fetus with trisomy 13, by determining the levels of at least two of sFlt-1, VEGF, or PlGF polypeptide in a sample from the subject and calculating the relationship between the levels of sFlt-1 VEGF, or PlGF using a metric. Desirably, the value obtained from the metric, known as the metric value, is compared to a reference and an alteration in the subject sample metric value relative to a reference metric value diagnoses a pregnant subject as having, or at risk for having, a fetus with trisomy 13. In preferred embodiments, the method also includes determining the body mass index (BMI), the gestational age (GA) of the fetus, or both and including the BMI or GA or both in the metric. In one embodiment, the metric is a pre-eclampsia anti-angiogenic index (PAAI): [sFlt-1/VEGF +PlGF], where the PAAI is used as an indicator of anti-angiogenic activity. In one embodiment, a PAAI greater than 10, more preferably greater than 20, is indicative of a pregnant subject as having, or at risk for having, a fetus with trisomy 13. In another embodiment, the metric is sFlt-1/PlGF and an sFlt-1/PlGF ratio greater than 15 diagnoses a pregnant subject as having, or at risk for having, a fetus with trisomy 13. For both the PAAI and the sFlt-1/PlGF, an increase in the metric value relative to a reference diagnoses a pregnant subject as having, or at risk for having, a fetus with trisomy 13. The sFlt-1/PlGF ratio can be determined at least two times between 11 weeks and 17 weeks. In normal pregnancy, the sFlt/PlGF ratio decreases during this time. An increase in the ratio or a lack of decrease in the ratio over this period diagnoses a pregnant subject as having, or at risk for having, a fetus with trisomy 13.

In preferred embodiments of the above aspects, the levels of sFlt- 1, VEGF, or PlGF polypeptide is determined by ELISA, preferably a sandwich ELISA, or a fluorescence immunoassay.

In another aspect, the invention provides a method of diagnosing a pregnant subject as having, or at risk for having, a fetus with trisomy 13 by measuring the level of sFlt-1, VEGF, or PlGF nucleic acid molecule in a sample from the subject and comparing it to a reference sample, where an alteration (e.g., increase or decrease of at least 10%, 20%, 30%, 40%, 50%, 60%, 70%, 80%, 90% or more) in the levels diagnoses the pregnant subject as having, or at risk for having a fetus with trisomy 13. Methods for detecting and quantitating nucleic acid molecules are known in the art and are described, for example, in Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning. A Laboratory Manual, Cold Spring Harbor Laboratory Press, New York.

In another aspect, the invention provides a method of diagnosing a pregnant subject as having, or at risk for having, a fetus with trisomy 13 by determining the nucleic acid sequence of a sFlt-1, VEGF, or PlGF gene in a subject and comparing it to a reference sequence, where an alteration in the subject's nucleic acid sequence that alters the level or the biological activity of the gene product in the subject diagnoses the pregnant subject as having, or at risk for having a fetus with trisomy 13 . In one embodiment, the alteration is a polymorphism in the nucleic acid sequence.

In various embodiments of the above aspects, the sample is a bodily fluid, such as blood, urine, amniotic fluid, serum, plasma, or cerebrospinal fluid, in which the sFlt-1, VEGF, or PlGF is normally detectable. In additional embodiments, the sample is a tissue or a cell. Non-limiting examples include placental tissue or placental cells, endothelial cells, leukocytes, and monocytes. In preferred embodiments of the above aspects, the level of sFlt-1 polypeptide measured is the level of free, bound, or total sFlt-1 polypeptide. In additional embodiments, the sFlt-1 polypeptide can also include sFlt-1 fragments, degradation products, or enzymatic cleavage products. In other preferred embodiments of the above aspects, the level of VEGF or PlGF is the level of free VEGF or free PlGF. In preferred embodiments, the subject is a human. In other embodiments of the above aspects, the subject is a non-human (e.g., a cow, a horse, a sheep, a pig, a goat, a dog, or a cat). The method can be carried out any time during pregnancy (e.g., first trimester, second trimester, or third trimester) but is preferably carried out during the first trimester, for example, at 4, 5, 6, 7, 8, 9, 10, 11, 12, or 13 weeks, or during the second trimester, for example at 14, 15, 16, 17, 18, 19, 20, 21, 22, 23, 24, 25, 26, or even 28 weeks.

In other embodiments of the above aspects, at least one of the levels measured is the level of sFlt-1 (free, bound, or total). In additional embodiments, the level of sFlt-1 measured includes the level of sFlt-1 degradation products or enzymatic cleavage products. In other embodiments of the above aspects, when the level of sFlt-1 is measured then the level of free PlGF is also measured. In additional embodiments, the BMI or GA or both is also measured. In various embodiments of the above aspects, an increase in the level of sFlt-1 nucleic acid or polypeptide relative to a reference diagnoses the pregnant subject as having, or at risk for having a fetus with trisomy 13. In other embodiments of the above aspects, a decrease in the level of free VEGF polypeptide or VEGF nucleic acid relative to a reference diagnoses the pregnant subject as having, or at risk for having a fetus with trisomy 13. In other embodiments of the above aspects, a decrease in the level of free PlGF polypeptide or PlGF nucleic acid relative to a reference diagnoses the pregnant subject as having, or at risk for having a fetus with trisomy 13.

In additional embodiments of the above aspects, the levels are measured on two or more occasions and a change in the levels between the measurements diagnoses the pregnant subject as having, or at risk for having, a fetus with trisomy 13. If the level of sFlt-1 increases from the first measurement to the next measurement or if the level of free VEGF or free PlGF decreases from the first measurement to the next measurement, this is considered diagnostic of fetal trisomy 13 or a risk for having a fetus with trisomy 13. The first occasion is desirably during the first trimester and the second occasion is during the second trimester.

In another aspect, the invention features a method of diagnosing a pregnant subject as having, or at risk for having, a fetus with trisomy 13 that includes measuring the level of free PlGF in a urine sample from the subject. This method can be used to determine absolute levels of free PlGF that are below a threshold level and diagnoses the subject as having, or at risk for having a fetus with trisomy 13. The normal urinary concentration of free PlGF is approximately 400-800 pg/ml during mid-pregnancy. In preferred embodiments, a level of free PlGF less than 400 pg/ml, preferably less than 300, 200, 100, 50, or 10 pg/ml is diagnostic of fetal trisomy 13 or a risk for having a fetus with trisomy 13. The levels of creatinine in a urine sample can also be measured and the absolute levels of free PlGF can be compared to the mg of creatinine present. This ratio allows for determination of PlGF concentration independent of dilution by the urine sample. This method can also be used to determine relative levels of free PlGF as compared to a reference sample where a decrease (e.g., 10%, 20%, 30%, 40%, 50%, 60%, 70%, 80%, 90%, or more) in the level of free PlGF as compared to a normal reference sample diagnoses a subject as having, or at risk for having, a fetus with trisomy 13. In this case, the normal reference sample can be a prior sample taken from the same subject or a sample taken from a matched subject (e.g., matched for gestational age) that is pregnant but does not have fetal trisomy 13, preferably as confirmed by genetic testing. In additional preferred embodiments, the reference sample is a standard, level, or number derived from such a normal reference sample. The reference standard or level can also be a value derived from a normal subject that is matched to the sample subject by at least one of the following criteria: gestational age of the fetus, age of the mother, blood pressure prior to pregnancy, blood pressure during pregnancy, BMI of the mother, weight of the fetus, prior diagnosis of pre-eclampsia or eclampsia or trisomy 13, and a family history of pre-eclampsia or eclampsia or trisomy 13. In preferred embodiments, the measuring is done using an immunological assay such as an ELISA, preferably a sandwich ELISA, or a fluorescence immunoassay.

In preferred embodiments, the method also includes measuring the level of at least one of sFlt-1, PlGF, and VEGF polypeptide in a sample from the subject, where the sample is a bodily fluid selected from the group consisting of urine, blood, amniotic fluid, serum, plasma, or cerebrospinal fluid. This method can be used alone to determine absolute levels as described above, or can be compared to the level of at least one of sFlt-1, PlGF, and VEGF in a reference sample. In preferred embodiments, an increase in the level of sFlt-1 or a decrease in the level of free VEGF or free PlGF polypeptide from the subject sample compared to the reference diagnoses the subject as having, or at risk for having, a fetus with trisomy 13. In preferred embodiments, sFlt-1 or sFlt-1 and PlGF are measured in a serum sample from a subject identified by a urine free PlGF assay as being at risk for fetal trisomy 13. In one example, the level of free PlGF in a sample of urine is first determined and then followed by measurement of sFlt-1 and/or free PlGF in a serum sample from the same subject. The ratio of sFlt-1/PlGF can be determined and the urine PlGF or the sflt-1/PlGF ratio or both can be used to diagnose a pregnant subject as having or at risk for having a fetus with trisomy 13.

Desirably, this method further includes calculating the relationship between the levels of at least two of sFlt-1, VEGF, and PlGF using a metric. This method can also be used alone or can be used to compare the metric value to a reference sample metric value, where an alteration in the subject sample metric value relative to the metric value in the reference sample diagnoses pre-eclampsia or eclampsia or a propensity to develop pre-eclampsia or eclampsia. Preferably, the metric is sFlt-1/PlGF or PAAI, where an increase in the sFlt-1/PlGF or PAAI value as compared to a normal reference or a prior sample from the same subject diagnoses a pregnant subject as having, or at risk for having a fetus with trisomy 13. In preferred embodiments, the sFlt-1 is free, bound, or total sFlt-1, and the PlGF and VEGF are free PlGF and free VEGF.

In another aspect, the invention features a method of diagnosing a pregnant subject as having, or at risk for having, a fetus with trisomy 13 that includes the following steps:

(a) obtaining a sample of urine from the subject;

(b) contacting the sample with a solid support, where the solid support includes an immobilized first PlGF binding agent, for a time sufficient to allow binding of the first PlGF binding agent with free PlGF present in the sample;

(c) contacting the solid support after step (b) with a preparation of a second labeled PlGF binding agent, for a time sufficient to allow binding of the second labeled PlGF binding agent to the free PlGF bound to the first immobilized PlGF binding agent;

(d) observing the binding of the second labeled PlGF binding agent to the immobilized PlGF binding agent bound to free PlGF at the position where the PlGF binding agent is immobilized; and

(e) comparing the binding observed in step (d) with the binding observed using a reference sample, where the reference sample is PlGF at a known concentration; and further where a decrease in the binding observed in step (d) compared to the binding observed using a reference sample diagnoses a pregnant subject as having, or at risk for having a fetus with trisomy 13.

In another related aspect, the invention features a method of diagnosing a pregnant subject as having, or at risk for having a fetus with trisomy 13 that includes the following steps:

(a) obtaining a urine sample from the subject;

(b) contacting the urine sample with a solid support, wherein the solid support comprises a dehydrated labeled PlGF binding agent and an immobilized secondary agent that binds the PlGF binding agent, for a time sufficient for the sample to rehydrate the labeled PlGF binding agent and to allow binding of free PlGF in the sample to the labeled PlGF binding agent, wherein the free PlGF bound to the labeled PlGF binding agent can move (e.g., by capillary movement) to the immobilized secondary agent;

(c) observing the binding of the free PlGF-PlGF binding agent complex to the immobilized secondary agent by detecting the presence of the label at the position where the secondary agent is immobilized; and

(d) comparing the binding observed in step (c) with the binding observed using a reference sample, wherein the reference sample is PlGF at known concentrations ranging from 10 pg/ml-1ng/ml.

In preferred embodiments of the above two aspects, the label is a colorimetric label (e.g., colloidal gold). In additional preferred embodiments, the agent that binds PlGF is an antibody, or purified fragment thereof, compound, or a peptide that specifically binds free PlGF. The agent that binds a PlGF agent is desirably an anti-immunoglobulin antibody or fragment thereof, protein A, or protein G. In one embodiment, the reference sample is a PlGF sample at a known normal concentration and a decrease in the free PlGF in the subject sample as compared to the reference sample subject as having, or at risk for having a fetus with trisomy 13.

In another aspect, the invention features a method of diagnosing a pregnant subject as having, or at risk for having a fetus with trisomy 13 that includes the following steps:

(a) obtaining a urine sample from the subject;

(b) contacting the sample with a solid support having an immobilized PlGF binding agent that is detectably labeled in a manner such that the label can distinguish between the PlGF when it is bound to free PlGF and when it is not bound to free PlGF. Preferred labels include fluorescent labels. The membrane is exposed to a urine sample obtained from the subject for a time sufficient to allow binding of the PlGF binding agent to free PlGF present in the sample. The labeled PlGF binding agent bound to the free PlGF is then measured. Such an assay can be used to determine the relative level of free PlGF (e.g., as compared to the level from a reference sample or standard or level) or to determine the absolute concentration of free PlGF as described above. Preferred assays for the measurement of binding include fluorescence immunoassays.

In another aspect the invention features a method of diagnosing a pregnant subject as having, or at risk for having a fetus with trisomy 13 that includes the following steps:

(a) obtaining a urine sample from the subject;

(b) contacting the sample with a solid support, wherein the solid support comprises an immobilized first PlGF binding agent, for a time sufficient to allow binding of the first PlGF binding agent with free PlGF present in the sample;

(c) contacting the solid support after step (b) with a preparation of a second PlGF binding agent coupled to an enzyme, for a time sufficient to allow binding of the second PlGF binding agent to the PlGF bound to the first immobilized PlGF binding agent; and

(d) adding a preparation of a substrate for the enzyme of step (c), for a time and in an amount sufficient to allow the enzyme to convert the substrate to a detectable substrate;

(e) observing the level of the detectable substrate; and

(f) comparing the level observed in step (e) with the binding observed using a reference sample, wherein the reference sample is PlGF at a known concentration, wherein an alteration in the level observed in step (e) as compared to the reference sample diagnoses a pregnant subject as having, or at risk for having, a fetus with trisomy 13.

In one embodiment, the reference sample is a PlGF sample at a known normal concentration and a decrease in the free PlGF in the subject sample as compared to the reference sample diagnoses the pregnant subject as having, or at risk for having, a fetus with trisomy 13.

In preferred embodiments of any of the above methods, the substrate is detected visually, by spectrophotometry or by chemiluminescence. In additional preferred embodiments, the enzyme is horseradish peroxidase, β-galactosidase, or alkaline phosphatase and the substrate is TMB (tetramethylbenzidine), Xgal (5-bromo-4-chloro-3-indolyl-β-D-galactopyranoside), or 1,2 dioxetane. In additional preferred embodiments, the reference sample is a sample having a normal concentration of purified PlGF and if the subject sample shows a decrease (10%, 20%, 30%, 40%, 50%, 60%, 70%, 80%, 90%, 95% or more) compared to the reference sample, the subject is diagnosed as having, or at risk for having a fetus with trisomy 13. In preferred embodiments of this method, the PlGF binding agent is a purified anti-PlGF antibody, or fragment thereof, compound, or peptide that specifically binds free PlGF.

In preferred embodiments of any of the above diagnostic methods, the solid support is a membrane that can be supported on a dipstick structure or a lateral flow format, examples of which are described in U.S. Pat. No. 6,660,534. In additional preferred embodiments, the subject is a pregnant human or a pregnant non-human (e.g., a cow, a horse, a sheep, a pig, a goat, a dog, or a cat). Desirably, the measuring of levels is done on two or more occasions and a change in the levels between measurements is a diagnostic indicator of pre-eclampsia or eclampsia. The method can be carried out any time during pregnancy (e.g., first, second, or third trimester) but is preferably carried out during the first trimester, for example, at 4, 5, 6, 7, 8, 9, 10, 11, 12, or 13 weeks, or during the second trimester, for example at 14, 15, 16, 17, 18, 19, 20, 21, 22, 23, 24, 25, 26, or even 28 weeks. For example, the levels of one or more of the polypeptides can be measured at first at about 10, 11, 12, or 13 weeks and then again at about 16, 17, 18, 19, or 20 weeks.

In additional preferred embodiment of any of the above diagnostic methods, the method also includes the measurement of any one, two or three of alpha-feto protein (AFP), human chorionic gonadotropin (hCG), and unconjugated estriol. The measurement of all three of these proteins in maternal serum samples is commonly known as the triple screen and is usually performed at 15 to 22 weeks (see, for example, Graves et al., Am. Fam. Physician 65:915-920 (2002)). Desirably, the same serum sample is used to measure the serum levels of sFlt-1, PlGF, or VEGF, or any combination thereof, and AFP, hCG, and unconjugated estriol. In another example, the diagnostic methods of the invention that feature measurement of free PlGF in a urine sample can be performed early in pregnancy and, if the level of PlGF is indicative of fetal trisomy 13, or a risk of having a fetus with trisomy 13, then the triple screen test can be performed in conjunction with the serum test for sFlt-1 or PlGF or both. When used in combination with additional tests such as the triple screen, the diagnostic methods of the invention, can be performed simultaneously or within 1 day, 2, days, 5 days, 1 week, 2 weeks, 3 weeks, 5 weeks, 10 weeks, 20 weeks, up to 30 or 35 weeks within each other.

In another aspect, the invention provides a kit for the diagnosis of fetal trisomy 13 or a risk of having a fetus with trisomy 13 in a pregnant subject that includes a component useful for detecting a sFlt-1, VEGF, or PlGF polypeptide, or any combination thereof. In preferred embodiments, the kit detects at least two of VEGF, sFlt-1, or PlGF. In additional preferred embodiments, the kit includes a binding agent (e.g., an antibody, or fragment or derivative thereof, or a peptide or compound) that specifically binds sFlt-1, VEGF, or PlGF, preferably free VEGF or free PlGF. The kit can also include components for an assay selected from the group consisting of an immunological assay, an enzymatic assay, and a calorimetric assay. In other preferred embodiments of the above aspects, when the kit includes components for the detection of sFlt-1, then components for the detection of free PlGF are also included. Optionally, the kit is used to determine the sFlt-1/PlGF ratio of the sample.

In another aspect, the invention provides a kit for the diagnosis of fetal trisomy 13 or a risk of having a fetus with trisomy 13 in a pregnant subject that includes a nucleic acid sequence, or fragment thereof, selected from the group consisting of sFlt-1, VEGF, and PlGF nucleic acid molecule, or a sequence complementary thereto, or any combination thereof, that specifically hybridizes to a sFlt-1, VEGF, or PlGF nucleic acid molecule. In a preferred embodiment, the kit comprises at least one nucleic acid probe, preferably at least two nucleic acid probes, for the detection of an sFlt-1, VEGF, or PlGF nucleic acid molecule.

The invention also a kit for the diagnosis of fetal trisomy 13 or a risk of having a fetus with trisomy 13 in a pregnant subject that includes a free PlGF binding agent for detecting free PlGF in a urine sample and instructions for its use for the diagnosis of fetal trisomy 13, or a risk of having a fetus with trisomy 13, in a pregnant subject. The kit can also include a component useful for an assay selected from the following: an immunological assay (e.g., an ELISA) an enzymatic assay or a colorimetric assay.

Desirably, the kits described above include any of the components needed to perform any of the diagnostic methods described above. For example, the kit desirably includes a membrane, where the free PlGF binding agent or the agent that binds the free PlGF binding agent is immobilized on the membrane. The membrane can be supported on a dipstick structure where the sample is deposited on the membrane by placing the dipstick structure into the sample or the membrane can be supported in a lateral flow cassette where the sample is deposited on the membrane through an opening in the cassette.

In preferred embodiments of any of the diagnostic kits described above, the diagnostic kits include a label or instructions for the intended use of the kit components and a reference sample or purified proteins to be used to establish a standard curve. In one embodiment, the diagnostic kit is labeled or includes instructions for use in diagnosis of fetal trisomy 13 or a risk of having a fetus with trisomy 13 in a pregnant subject. The diagnostic kit can also include a label or instructions for the use of the kit to determine the PAAI or sFlt-1/PlGF of the subject sample and to compare the PAAI or sFlt-1/PlGF to a reference sample value. It will be understood that the reference sample values will depend on the intended use of the kit. For example, the sample can be compared to a normal PAAI reference value, a normal sFlt-1/PlGF ratio, or a normal free PlGF value, wherein an increase in the PAAI, sFlt1-/PlGF, or a decrease in the PlGF value diagnoses fetal trisomy 13 or a risk of having a fetus with trisomy 13 in a pregnant subject. In another preferred embodiment, the diagnostic kit includes a label or instructions for the use of the kit to determine the sFlt-1/PlGF ratio of the subject sample and to compare the sFlt-1/PlGF ratio to a reference sample value.

In additional preferred embodiments of any of the kits of the invention, the kit can also include components for the measurement of any one, two or three of alph-feto protein (AFP), human chorionic gonadotropin (hCG), and unconjugated estriol. The measurement of all three of these proteins in maternal serum samples is commonly known as the triple screen and is usually performed at 15 to 22 weeks (see, for example, Graves et al., Am. Fam. Physician 65:915-920 (2002)).

Any of the methods and kits described herein can be used for the diagnosis of fetal trisomy 13 or a risk of having a fetus with trisomy 13 or to identify women who should undergo further testing for genetic abnormalities such as trisomy 13.

In another related aspect, the invention features a device for diagnosing fetal trisomy 13, or a risk of having a fetus with trisomy 13, in a pregnant subject that includes a component for comparing the levels of at least one of sFlt-1, VEGF, and PlGF polypeptides in a sample from a subject relative to a reference sample, wherein an alteration (e.g., 10%, 20%, 30%, 40%, 50%, 60%, 70%, 80%, 90%, or more) in the levels of at least one of sFlt-1, VEGF, and PlGF is a diagnostic indicator of fetal trisomy 13, or a risk of having a fetus with trisomy 13, in a pregnant subject. In a preferred embodiment, the device includes a component for using a metric to compare the levels as at least two of sFlt-1, VEGF, and PlGF polypeptides.

In a related aspect, the invention features a device for diagnosing fetal trisomy 13 or a risk of having a fetus with trisomy 13 in a pregnant subject that includes a component for comparing the levels of at least one of sFlt-1, VEGF, and PlGF nucleic acid molecules in a sample from a subject relative to a reference sample, wherein an alteration in the levels of at least one of sFlt-1, VEGF, and PlGF fetal trisomy 13 or a risk of having a fetus with trisomy 13 in the subject. In a preferred embodiment the device includes a component for using a metric to compare the levels as at least two of sFlt-1, VEGF, and PlGF nucleic acid molecules.

For the purpose of the present invention, the following abbreviations and terms are defined below.

By “alteration” is meant a change (increase or decrease). An alteration can be a change in the expression levels of a gene or polypeptide as detected by standard art known methods such as those described above. As used herein, an alteration includes a 10%, 20%, 30%, 40%, 50%, 60%, 70%, 80%, 90% or greater change in expression levels. “Alteration” can also indicate a change (increase or decrease) in the biological activity of any of the polypeptides of the invention (e.g., sFlt-1, VEGF, or PlGF). As used herein, an alteration includes 10%, 20%, 30%, 40%, 50%, 60%, 70%, 80%, 90% or greater change in biological activity. Examples of biological activity for PlGF or VEGF include binding to receptors as measured by immunoassays, ligand binding assays or Scatchard plot analysis, and induction of cell proliferation or migration as measured by BrdU labeling, cell counting experiments, or quantitative assays for DNA synthesis such as ³H-thymidine incorporation. Examples of biological activity for sFlt-1 include binding to PlGF and VEGF as measured by immunoassays, ligand binding assays, or Scatchard plot analysis. Additional examples of assays for biological activity for each of the polypeptides are described herein.

By “antisense nucleobase oligomer” is meant a nucleobase oligomer, regardless of length, that is complementary to the coding strand or mRNA of an sFlt-1 gene. By a “nucleobase oligomer” is meant a compound that includes a chain of at least eight nucleobases, preferably at least twelve, and most preferably at least sixteen bases, joined together by linkage groups. Included in this definition are natural and non-natural oligonucleotides, both modified and unmodified, as well as oligonucleotide mimetics such as Protein Nucleic Acids, locked nucleic acids, and arabinonucleic acids. Numerous nucleobases and linkage groups may be employed in the nucleobase oligomers of the invention, including those described in U.S. Patent Application Publication Nos. 20030114412 and 20030114407, incorporated herein by reference. The nucleobase oligomer can also be targeted to the translational start and stop sites. Preferably the antisense nucleobase oligomer comprises from about 8 to 30 nucleotides. The antisense nucleobase oligomer can also contain at least 40, 60, 85, 120, or more consecutive nucleotides that are complementary to sFlt-1 mRNA or DNA, and may be as long as the full-length mRNA or gene.

By “body mass index” is meant a number, derived by using height and weight measurements, that gives a general indication of whether or not weight falls within a healthy range. The formula generally used to determine the body mass index is a person's weight in kilograms divided by a person's height in meters squared or weight (kg)/(height (m))².

By “compound” is meant any small molecule chemical compound, antibody, nucleic acid molecule, or polypeptide, or fragments thereof.

By “expression” is meant the detection of a gene or polypeptide by standard art known methods. For example, polypeptide expression is often detected by western blotting, DNA expression is often detected by Southern blotting or polymerase chain reaction (PCR), and RNA expression is often detected by northern blotting, PCR, or RNAse protection assays.

By “fragment” is meant a portion of a polypeptide or nucleic acid molecule. A fragment may contain 10, 20, 30, 40, 50, 60, 70, 80, 90, or 100, 200, 300, 400, 500, 600, 700, 800, 900, or 1000 or more nucleotides or amino acids. A fragment can also mean a portion that includes at least 10%, 20%, 30%, 40%, 50%, 60%, 70%, 80%, or 90% of the entire length of the nucleic acid molecule or polypeptide.

By “gestational age” is meant a reference to the age of the fetus, counting from the first day of the mother's last menstrual period and is usually referred to in weeks.

By a “history of pre-eclampsia or eclampsia” is meant a previous diagnosis of pre-eclampsia or eclampsia or pregnancy-induced hypertension in the subject themselves or in a related family member.

By “homologous” is meant any gene or polypeptide sequence that bears at least 30% homology, more preferably 40%, 50%, 60%, 70%, 80%, and most preferably 90% or more homology to a known gene or polypeptide sequence over the length of the comparison sequence. A “homologous” polypeptide can also have at least one biological activity of the comparison polypeptide. For polypeptides, the length of comparison sequences will generally be at least 6 amino acids, preferably at least 10 or 20 amino acids, more preferably at least 25 amino acids, and most preferably 50, 100, 150, 200 amino acids or more, up to the entire length of the polypeptide. For nucleic acids, the length of comparison sequences will generally be at least 18 nucleotides, preferably at least 25 or 50 nucleotides, more preferably at least 75 nucleotides, and most preferably from at least 100, 150, 200, 250, 300 nucleotides or more up to the entire length of the nucleic acid. “Homology” can also refer to a substantial similarity between an epitope used to generate antibodies and the polypeptide or fragment thereof to which the antibodies are directed. In this case, homology refers to a similarity sufficient to elicit the production of antibodies that can specifically recognize the polypeptide at issue.

By “hybridize” is meant pair to form a double-stranded molecule between complementary polynucleotide sequences, or portions thereof, under various conditions of stringency. (See, e.g., Wahl and Berger (1987) Methods Enzymol. 152:399; Kimmel, Methods Enzymol. 152:507, 1987.) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning. A Laboratory Manual, Cold Spring Harbor Laboratory Press, New York.

By “intrauterine growth retardation (IUGR)” is meant a syndrome resulting in a birth weight which is less that 10 percent of the predicted fetal weight for the gestational age of the fetus. The current World Health Organization criterion for low birth weight is a weight less than 2,500 gm (5 lbs. 8 oz.) or below the 10^{th} percentile for gestational age according to U.S. tables of birth weight for gestational age by race, parity, and infant sex (Zhang and Bowes, Obstet. Gynecol. 86:200-208, 1995). These low birth weight babies are also referred to as “small for gestational age (SGA).” Pre-eclampsia is a condition known to be associated with IUGR or SGA.

By “metric” is meant a measure. A metric may be used, for example, to compare the levels of a polypeptide or nucleic acid molecule of interest. Exemplary metrics include, but are not limited to, mathematical formulas or algorithms, such as ratios. The metric to be used is that which best discriminates between levels of sFlt-1, VEGF, or PlGF in a subject having, or at risk for having, a fetus with trisomy 13 and a normal control subject. Depending on the metric that is used, the diagnostic indicator of a subject having, or at risk for having, a fetus with trisomy 13, may be significantly above or below a reference value (e.g., from a control subject not having fetal trisomy 13). The metric can also include the BMI or the GA for the subject. sFlt-1 level is generally measured by measuring the amount of free, bound (i.e., bound to growth factor), or total sFlt-1 (bound+free). VEGF or PlGF levels are generally determined by measuring the amount of free PlGF or free VEGF (i.e., not bound to sFlt-1). One exemplary metric is [sFlt-1/(VEGF+PlGF)], also referred to as the pre-eclampsia anti-angiogenic index (PAAI). Another exemplary metric is sFlt-1/PlGF. In this example, sFlt-1 and PlGF are preferably detected in a maternal serum sample.

By “operably linked” is meant that a gene and a regulatory sequence(s) are connected in such a way as to permit gene expression when the appropriate molecules (e.g., transcriptional activator proteins) are bound to the regulatory sequence(s).

By “placental growth factor (PlGF)” is meant a mammalian growth factor that is homologous to the protein defined by GenBank accession number P49763 and that has PlGF biological activity. PlGF is a glycosylated homodimer belonging to the VEGF family and can be found in two distinct isoforms through alternative splicing mechanisms. PlGF is expressed by cyto- and syncytiotrophoblasts in the placenta and PlGF biological activities include induction of proliferation, migration, and activation of endothelial cells, particularly trophoblast cells.

By “polymorphism” is meant a genetic variation, mutation, deletion or addition in an sFlt-1, PlGF, or VEGF nucleic acid molecule that is indicative of a predisposition to develop the conditions. Such polymorphisms are known to the skilled artisan and are described by Parry et al. (Eur. J Immunogenet. 26:321-3, 1999). A polymorphism may be present in the promoter sequence, an open reading frame, intronic sequence, or untranslated 3′ region of an sFlt-1 gene.

By “pre-eclampsia” is meant the multi-system disorder that is characterized by hypertension with proteinuria or edema, or both, glomerular dysfunction, brain edema, liver edema, or coagulation abnormalities due to pregnancy or the influence of a recent pregnancy. Pre-eclampsia generally occurs after the 20^{th} week of gestation. Pre-eclampsia is generally defined as some combination of the following symptoms: (1) a systolic blood pressure (BP)>140 mmHg and a diastolic BP>90 mmHg after 20 weeks gestation (generally measured on two occasions, 4-168 hours apart), (2) new onset proteinuria (1+ by dipstik on urinanaysis, >300 mg of protein in a 24-hour urine collection, or a single random urine sample having a protein/creatinine ratio >0.3), and (3) resolution of hypertension and proteinuria by 12 weeks postpartum. Severe pre-eclampsia is generally defined as (1) a diastolic BP>110 mmHg (generally measured on two occasions, 4-168 hours apart) or (2) proteinuria characterized by a measurement of 3.5 g or more protein in a 24-hour urine collection or two random urine specimens with at least 3+ protein by dipstick. In pre-eclampsia, hypertension and proteinuria generally occur within seven days of each other. In severe pre-eclampsia, severe hypertension, severe proteinuria and HELLP syndrome (hemolysis, elevated liver enzymes, low platelets) or eclampsia can occur simultaneously or only one symptom at a time. Occasionally, severe pre-eclampsia can lead to the development of seizures. This severe form of the syndrome is referred to as “eclampsia.” Eclampsia can also include dysfunction or damage to several organs or tissues such as the liver (e.g., hepatocellular damage, periportal necrosis) and the central nervous system (e.g., cerebral edema and cerebral hemorrhage). The etiology of the seizures is thought to be secondary to the development of cerebral edema and focal spasm of small blood vessels in the kidney.

By “pre-eclampsia anti-angiogenesis index (PAAI)” is meant the ratio of sFlt-1/VEGF+PlGF used as an indicator of anti-angiogenic activity. A PAAI greater than 10, more preferably greater than 20, is considered to be indicative of pre-eclampsia or risk of pre-eclampsia.

By “probe” is meant a single-stranded DNA or RNA molecule of defined sequence that can base pair to a second DNA or RNA molecule that contains a complementary sequence (“target”). The stability of the resulting hybrid depends upon the extent of the base pairing that occurs. This stability is affected by parameters such as the degree of complementarity between the probe and target molecule, and the degree of stringency of the hybridization conditions. The degree of hybridization stringency is affected by parameters such as the temperature, salt concentration, and concentration of organic molecules, such as formamide, and is determined by methods that are well known to those skilled in the art. Probes that specifically bind to or hybridize to sFlt-1, PlGF or VEGF nucleic acid molecules, preferably, have greater than 45% sequence identity, more preferably at least 55-75% sequence identity, still more preferably at least 75-85% sequence identity, yet more preferably at least 85-99% sequence identity, and most preferably 100% sequence identity to the nucleic acid sequences encoding the amino acid sequences described herein. Probes can be detectably-labeled, either radioactively or non-radioactively, by methods that are well-known to those skilled in the art. Probes can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), and other methods that are well known to those skilled in the art.

By “protein” or “polypeptide” or “polypeptide fragment” is meant any chain of more than two amino acids, regardless of post-translational modification (e.g., glycosylation or phosphorylation), constituting all or part of a naturally occurring polypeptide or peptide, or constituting a non-naturally occurring polypeptide or peptide.

By “reduce or inhibit” is meant the ability to cause an overall decrease preferably of 10%, 20%, 30%, 40%, 50%, 60%, 70%, 80%, 90%, 95%, 96%, 97%, 98%, 99% or more in the level of polypeptide or nucleic acid, detected by the aforementioned assays (see “expression”) or the biological activity of the polypeptide, detected by the aforementioned assays (see “biological activity”), as compared to a reference sample.

By “reference sample” is meant any sample, standard, or level that is used for comparison purposes. A “normal reference sample” can be a prior sample taken from the subject, a sample taken from a pregnant subject known to have a normal fetus (e.g., as confirmed by amniocentesis, FISH, CVS or other methods for detecting genetic abnormalities), a subject that is pregnant but the sample was taken early in pregnancy (e.g., in the first or second trimester or before the detection of trisomy 13), a subject that is pregnant but does not have a fetus with trisomy 13 (e.g., as confirmed by amniocentesis or CVS or other methods for detecting genetic abnormalities) and has no history of fetal trisomy 13. By “reference standard or level” is meant a value or number derived from a reference sample. A normal reference standard or level can be a value or number derived from a normal subject that is matched to the sample subject by at least one of the following criteria: gestational age of the fetus, maternal age, maternal blood pressure prior to pregnancy, maternal blood pressure during pregnancy, BMI of the mother, weight of the fetus, prior diagnosis of pre-eclampsia or eclampsia, prior diagnosis of fetal trisomy 13, and a family history of pregnancy related hypertensive disorders, such as pre-eclampsia or eclampsia. A “positive reference” sample, standard or value is a sample or value or number derived from a subject that is known to have a trisomy 13 fetus, or a subject that has or is at high risk for the development of pre-eclampsia such as prior history of pre-eclampsia, preexisting diabetes, preexisting hypertension, pregnancy complicated by multiple gestation or molar pregnancy. In each case, the reference sample is preferably matched to the sample subject by at least one of the following criteria: gestational age of the fetus, maternal age, maternal blood pressure prior to pregnancy, maternal blood pressure during pregnancy, BMI of the mother, weight of the fetus, prior diagnosis of pre-eclampsia, eclampsia, or fetal trisomy 13, and a family history of fetal trisomy 13. A normal reference sample can also be a purified polypeptide (e.g., PlGF, VEGF, or sFlt-1) at a concentration known to be a normal concentration not diagnostic of fetal trisomy 13 or a pregnant subject at risk of having a fetus with trisomy 13. For example, serum free PlGF concentrations during normal pregnancy may range from 400-800 pg/ml, whereas those with fetal trisomy 13 may be below 200 pg/ml, preferably below 150 pg/ml, during mid-gestation. A level of urinary PlGF below 400 pg/ml or below 200 pg/ml may be indicative of fetal trisomy 13 or a subject at risk of having a fetus with trisomy 13. A reference value can also be used which is determined based on the values of a particular polypeptide in a reference sample.

By “sample” is meant a tissue biopsy, cell, bodily fluid (e.g., blood, serum, plasma, urine, saliva, amniotic fluid, or cerebrospinal fluid) or other specimen obtained from a subject. Desirably, the biological sample includes polypeptides of the invention or nucleic acid molecules encoding polypeptides of the invention or both.

By “soluble Flt-1 (sFlt-1)” (also known as sVEGF-R1) is meant the soluble form of the Flt-1 receptor, that is homologous to the protein defined by GenBank accession number U01134, and that has sFlt-1 biological activity. The biological activity of an sFlt-1 polypeptide may be assayed using any standard method, for example, by assaying sFlt-1 binding to VEGF. sFlt-1 lacks the transmembrane domain and the cytoplasmic tyrosine kinase domain of the Flt-1 receptor. sFlt-1 can bind to VEGF and PlGF with high affinity, but it cannot induce proliferation or angiogenesis and is therefore functionally different from the Flt-1 and KDR receptors. sFIt-1 was initially purified from human umbilical endothelial cells and later shown to be produced by trophoblast cells in vivo. As used herein, sFlt-1 includes any sFlt-1 family member or isoform. In additional embodiments, sFlt-1 can also mean degradation products or fragments that result from enzymatic cleavage of the Flt-1 receptor and that maintain sFlt-1 biological activity. In one example, specific metalloproteinases released from the placenta may cleave the extracellular domain of Flt-1 receptor to release the N-terminal portion of Flt-1 into circulation.

By “specifically binds” is meant any compound, nucleic acid, polypeptide (e.g., an antibody) which recognizes and binds a polypeptide or nucleic acid molecule of the invention but that does not substantially recognize and bind other molecules in a sample, for example, a biological sample, which naturally includes a polypeptide of the invention. In one example, an antibody that specifically binds sFlt-1 does not bind Flt-1. In another example, an antibody that specifically binds free PlGF does not bind bound PlGF.

By “subject” is meant a mammal, including, but not limited to, a human or non-human mammal, such as a bovine, equine, canine, ovine, or feline. Included in this definition are pregnant, post-partum, and non-pregnant mammals.

By “substantially identical” is meant a nucleic acid or amino acid sequence that, when optimally aligned, for example using the methods described below, share at least 60%, 65%, 70%, 75%, 80%, 85%, 90%, 95%, 96%, 97%, 98%, 99%, or 100% sequence identity with a second nucleic acid or amino acid sequence, e.g., an endoglin or soluble endoglin sequence. “Substantial identity” may be used to refer to various types and lengths of sequence, such as full-length sequence, epitopes or immunogenic peptides, functional domains, coding and/or regulatory sequences, exons, introns, promoters, and genomic sequences. Percent identity between two polypeptides or nucleic acid sequences is determined in various ways that are within the skill in the art, for instance, using publicly available computer software such as Smith Waterman Alignment (Smith, T. F. and M. S. Waterman (1981) J. Mol. Biol. 147:195-7); “Best Fit” (Smith and Waterman, Advances in Applied Mathematics, 482-489 (1981)) as incorporated into GeneMatcher Plus™, Schwarz and Dayhof (1979) Atlas of Protein Sequence and Structure, Dayhof, M. O., Ed pp 353-358; BLAST program (Basic Local Alignment Search Tool; (Altschul, S. F., W. Gish, et al. (1990) J. Mol. Biol. 215: 403-10), BLAST-2, BLAST-P, BLAST-N, BLAST-X, WU-BLAST-2, ALIGN, ALIGN-2, CLUSTAL, or Megalign (DNASTAR) software. In addition, those skilled in the art can determine appropriate parameters for measuring alignment, including any algorithms needed to achieve maximal alignment over the length of the sequences being compared. In general, for proteins, the length of comparison sequences will be at least 6 amino acids, preferably 10, 20, 30, 40, 50, 60, 70, 80, 90, 100, 110, 120, 130, 140, 150, 200, 250, 300, 350, 400, or 500 amino acids or more up to the entire length of the protein. For nucleic acids, the length of comparison sequences will generally be at least 18, 25, 50, 100, 125, 150, 200, 250, 300, 350, 400, 450, 500, 550, 600, 650, 700, 800, 900, 1000, 1100, 1200, or at least 1500 nucleotides or more up to the entire length of the nucleic acid molecule. It is understood that for the purposes of determining sequence identity when comparing a DNA sequence to an RNA sequence, a thymine nucleotide is equivalent to a uracil nucleotide. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

By “symptoms of pre-eclampsia” is meant any of the following: (1) a systolic blood pressure (BP)>140 mmHg and a diastolic BP>90 mmHg after 20 weeks gestation, (2) new onset proteinuria (1+ by dipstik on urinanaysis, >300 mg of protein in a 24 hour urine collection, or random urine protein/creatinine ratio>0.3), and (3) resolution of hypertension and proteinuria by 12 weeks postpartum. The symptoms of pre-eclampsia can also include renal dysfunction and glomerular endotheliosis or hypertrophy. By “symptoms of eclampsia” is meant the development of any of the following symptoms due to pregnancy or the influence of a recent pregnancy: seizures, coma, thrombocytopenia, liver edema, pulmonary edema, and cerebral edema.

By “trisomy 13” is meant a condition characterized by the presence of three copies of chromosome 13. Trisomy 13 can result in a syndrome characterized by mental retardation and defects to the central nervous system and heart. Trisomy 13 can also involve conditions characterized by duplications of only portions of the chromosome 13, for example only the short arm or long arm of chromosome 13. These partial duplications are sometimes referred to as partial trisomy 13.

By “trophoblast” is meant the mesectodermal cell layer covering the blastocyst that erodes the uterine mucosa and through which the embryo receives nourishment from the mother; the cells contribute to the formation of the placenta.

By “vascular endothelial growth factor (VEGF)” is meant a mammalian growth factor that is homologous to the growth factor defined in U.S. Pat. Nos. 5,332,671; 5,240,848; 5,194,596; and Charnock-Jones et al. (Biol. Reproduction, 48: 1120-1128, 1993), and has VEGF biological activity. VEGF exists as a glycosylated homodimer and includes at least four different alternatively spliced isoforms. The biological activity of native VEGF includes the promotion of selective growth of vascular endothelial cells or umbilical vein endothelial cells and induction of angiogenesis. As used herein, VEGF includes any VEGF family member or isoform (e.g. VEGF-A, VEGF-B, VEGF-C, VEGF-D, VEGF-E, VEGF189, VEGF165, or VEGF 121). Preferably, VEGF is the VEGF121 or VEGF165 isoform (Tischer et al., J. Biol. Chem. 266, 11947-11954, 1991; Neufed et al. Cancer Metastasis 15:153-158, 1996), which is described in U.S. Pat. Nos. 6,447,768; 5,219,739; and 5,194,596, hereby incorporated by reference. Also included are mutant forms of VEGF such as the KDR-selective VEGF and Flt-selective VEGF described in Gille et al. (J. Biol. Chem. 276:3222-3230, (2001)). As used herein VEGF also includes any modified forms of VEGF such as those described in LeCouter et al. (Science 299:890-893, 2003). Although human VEGF is preferred, the invention is not limited to human forms and can include other animal forms of VEGF (e.g. mouse, rat, dog, or chicken).

By “vector” is meant a DNA molecule, usually derived from a plasmid or bacteriophage, into which fragments of DNA may be inserted or cloned. A recombinant vector will contain one or more unique restriction sites, and may be capable of autonomous replication in a defined host or vehicle organism such that the cloned sequence is reproducible. A vector contains a promoter operably linked to a gene or coding region such that, upon transfection into a recipient cell, an RNA is expressed.

Other features and advantages of the invention will be apparent from the following description of the preferred embodiments thereof, and from the claims.

### BRIEF DESCRIPTION OF THE DRAWINGS

FIG. 1A is a graph showing the mean concentrations of serum circulating sFlt-1 and PlGF in pg/ml in normal karyotype controls (n=85), trisomy 13 (n=17), trisomy 18 (n=20) and trisomy 21 (n=17) patients. sFlt1 and PlGF levels for the various trisomy groups were compared to the normal karyotype controls. P value for PlGF measurements between trisomy 13 as compared to control was significant at 0.032; all other comparisons were not significant.

FIG. 1B is a box plot with whiskers of the ratio sFlt-1/PlGF in normal karyotype controls (n=85), trisomy 13 (n=17), trisomy 18 (n=20) and trisomy 21 (n=17) patients. The bold line represents the median, and the box and whiskers with the interquartile range is presented. The points beyond the whiskers represent outliers. * * * represents P=0.003 as compared to controls.

FIG. 2 is a box plot with whiskers of the ratio sFlt-1/PlGF in normal karyotype controls controls (n=30 and n=55) and trisomy 13 (n=6 and n=11) in first and second trimester serum specimens respectively. The bold line represents the median, and the box and whiskers with the interquartile range is presented. The points beyond the whiskers represent outliers. * * * represents P=0.007 in the trisomy 13 comparisons with the controls.

### DETAILED DESCRIPTION

We have discovered that serum levels of sFlt-1 are increased in women carrying trisomy 13 fetuses, possible due to the extra copy of the placental Flt-1 gene. We have also discovered that in women carrying trisomy 13 fetuses, there is a decrease in the level of PlGF and an increase in the level of the sFlt-1/PlGF ratio. The most immediate clinical ramification of our discovery is that levels of sFlt-1, PlGF, and VEGF, as well as the ratio of sFlt-1/PlGF can be used to diagnose the likelihood of fetal trisomy 13 in a pregnant subject.

We have previously discovered that sFlt-1 levels are elevated in blood serum samples taken from pre-eclamptic women. sFlt-1 binds to VEGF and PlGF with high affinity and blocks the mitogenic and angiogenic activity of these growth factors. Thus, sFlt-1, VEGF, and PlGF are useful both as diagnostic makers and therapeutic targets for pre-eclampsia and eclampsia. We have also previously discovered that PlGF levels in the urine can be used as a diagnostic tool to detect pre-eclampsia or eclampsia, or a predisposition thereto. The free form of PlGF has an average molecular weight of about 30 kDa and is small enough to be filtered by the kidney and released into the urine. PlGF, when complexed to sFlt-1, has a much greater molecular weight and would therefore not be released into the urine. When the levels of sFlt-1 are increased, sFlt-1 can complex to PlGF, thereby reducing the levels of free PlGF released into the urine. As a result, urine analysis for free PlGF levels can be used to diagnose pre-eclampsia or eclampsia or a patient at risk for having the same. These discoveries are described in U.S. patent application publication Nos. 20040126828, 20050025762, and 20050170444, and PCT application publication Nos. WO2004/008946 and WO2005/077007, each of which is herein incorporated by reference in their entirety.

sFlt-1, a splice variant of the Flt-1 gene is encoded on the long arm of chromosome 13, specifically in the 13q12 region. We have discovered that serum levels of sFlt-1 are elevated in women carrying trisomy 13 fetuses, possible due to the extra copy of the placental Flt-1 gene. We have also discovered that in women carrying trisomy 13 fetuses, there is a decrease in the level of free PlGF and in increase in the sFlt-1/PlGF ratio, suggesting that the alteration in the circulating angiogenic state may be responsible for the increased risk of pre-eclampsia observed in these patients.

While the detailed description presented herein refers specifically to sFlt-1, VEGF, or PlGF, it will be clear to one skilled in the art that the detailed description can also apply to sFlt-1, VEGF, or PlGF family members, isoforms, and/or variants, and to any additional growth factors shown to bind sFlt-1.

Diagnostics

The present invention features diagnostic assays for the detection of fetal trisomy in a pregnant subject, or a pregnant subject's risk of having a fetus with trisomy 13. Levels of VEGF or PlGF (preferably free VEGF or PlGF) or sFlt-1 polypeptide, either free or total levels, are measured in a sample from a pregnant subject sample and used as an indicator of fetal trisomy 13 or a risk of having a fetus with trisomy 13.

Elevated levels of sFlt-1 and decreased levels of PlGF or VEGF in a sample of from a pregnant subject are considered a positive indicator of a fetus with trisomy 13 or a risk of having a fetus with trisomy 13. For example, an sFlt-1 serum value of 2 ng/ml or greater is considered a positive indicator of a fetus with trisomy 13 or a risk of having a fetus with trisomy 13. The sFlt-1 polypeptide can include full-length sFlt-1, degradation products, alternatively spliced isoforms of sFlt-1, enzymatic cleavage products of sFlt-1, and the like. A serum free PlGF value of less than 50 pg/ml at 10-12 weeks, less than 100 pg/ml at 13-16 weeks, less than 200 pg/ml at 17-20 weeks, less than 300 pg/ml at 21-24 weeks, 25-28 weeks, 29-32 weeks, or 33-37 weeks, and less than 250 pg/ml at 37 to 41 weeks is considered a positive indicator of a fetus with trisomy 13 or a risk of having a fetus with trisomy 13. Any binding agent (e.g., polypeptide, antibody, or compound) that specifically binds an sFlt-1, VEGF, or PlGF (preferably free VEGF or free PlGF) polypeptide may be used for the diagnosis of a fetus with trisomy 13 or a risk of having a fetus with trisomy 13 in a pregnant subject. A variety of protocols for measuring an alteration in the expression of such polypeptides are known, including immunological methods (such as ELISAs and RIAs), and provide a basis for diagnosing a fetus with trisomy 13 or a risk of having a fetus with trisomy 13 in a pregnant subject. Additionally, any detectable alteration (e.g., an increase or decrease) in levels of sFlt-1, VEGF, or PlGF, or any combination thereof, relative to a normal reference level is indicative of a fetus with trisomy 13 or a risk of having a fetus with trisomy 13 in a pregnant subject. Preferably, sFlt-1 is measured, more preferably measurement of free PlGF is combined with this measurement. In additional preferred embodiments, the body mass index (BMI) and gestational age of the fetus is also measured and included the diagnostic metric.

Standard methods may be used to measure levels of VEGF, PlGF, or sFlt-1 polypeptide in any bodily fluid, including, but not limited to, urine, serum, plasma, saliva, amniotic fluid, or cerebrospinal fluid. Such methods include immunoassay, ELISA, “sandwich assays”, western blotting using antibodies directed to VEGF, PlGF or sFlt-1, immunodiffusion assays, agglutination assays, fluorescent immunoassays, protein A or G immunoassays, and immunoelectrophoresis assays and quantitative enzyme immunoassay techniques such as those described in Ong et al. (Obstet. Gynecol. 98:608-611, 2001) and Su et al. (Obstet. Gynecol., 97:898-904, 2001). ELISA assays are the preferred method for measuring levels of VEGF, PlGF, or sFlt-1. Particularly preferred, for ease and simplicity of detection, and its quantitative nature, is the sandwich or double antibody assay of which a number of variations exist, all of which are contemplated by the present invention. For example, in a typical sandwich assay, unlabeled antibody that specifically binds the antigen (i.e., sFlt-1, free PlGF, or free VEGF polypeptide) is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

In one embodiment, at least two of the proteins are measured and a metric is used to determine whether a relationship between the levels is indicative of a fetus with trisomy 13 or a risk of having a fetus with trisomy 13. One example of a metric is the PAAI (sFlt-1/ VEGF+PlGF), which can be used to identify a subject having a fetus with trisomy 13 or at risk of having a fetus with trisomy 13. If the PAAI is greater than 10, more preferably greater than 20, then the subject is identified as having a fetus with trisomy 13 or at risk of having a fetus with trisomy 13. Another example of a useful metric is the sFlt-1/PlGF ratio. If the sFlt-1/PlGF ratio is greater than 15, then the subject is identified as having a fetus with trisomy 13 or at risk of having a fetus with trisomy 13. If the PAAI or the sFlt-1/PlGF ratio is increased (e.g., by at least 10%, 20%, 30%, 40%, 50%, 60%, 70%, 80%, 90%, or more) as compared to the ratio in a normal reference sample, then the pregnant subject is considered to have a fetus with trisomy 13 or to be at risk of having a fetus with trisomy 13. Virtually any metric that detects an alteration in the levels of any of sFlt-1, PlGF, or VEGF in a subject relative to a normal control may be used as a diagnostic indicator of a pregnant subject having a fetus with trisomy 13 or at risk of having a fetus with trisomy 13.

Oligonucleotides or longer fragments derived from a sFlt-1, PlGF, or VEGF nucleic acid sequence may be used as a probe to detect alterations in the expression of these nucleic acids as compared to a normal reference subject sample and correlated to the presence of a fetus with trisomy 13 in a pregnant subject or a risk of having a fetus with trisomy 13. Such probes can also be used to identify subjects having a genetic variation, mutation, or polymorphism in an sFlt-1, PlGF, or VEGF nucleic acid molecule that are indicative of a a fetus with trisomy 13 in a pregnant subject or a risk of having a fetus with trisomy 13. Such polymorphisms are known to the skilled artisan and are described, for example, by Parry et al. (Eur. J Immunogenet. 26:321-3, 1999). A survey of the GenBank database (www.ncbi.nlm.nih.gov) reveals at least 330 known polymorphisms in the gene and the promoter region of Flt-1/sFlt-1. These polymorphisms may affect sFlt-1 nucleic acid or polypeptide expression levels or biological activity. Detection of genetic variation, mutation, or polymorphism relative to a normal, reference sample can be as a diagnostic indicator of a fetus with trisomy 13 or a risk of having a fetus with trisomy 13 in a pregnant subject.

Such genetic alterations may be present in the promoter sequence, an open reading frame, intronic sequence, or untranslated 3′ region of an sFlt-1 gene. Information related to genetic alterations can be used to diagnose a subject as having a fetus with trisomy 13 in a pregnant subject or a risk of having a fetus with trisomy 13. As noted throughout, specific alterations in the levels of biological activity of sFlt-1, VEGF, and/or PlGF can be correlated with the likelihood of a fetus with trisomy 13 in a pregnant subject or a risk of having a fetus with trisomy 13. As a result, one skilled in the art, having detected a given mutation, can then assay one or more metrics of the biological activity of the protein to determine if the mutation causes or increases the likelihood of having a fetus with trisomy 13 in a pregnant subject.

In one embodiment, a subject having a fetus with trisomy 13 or a risk of having a fetus with trisomy 13 will show an increase (e.g., 10%, 20%, 30%, 40%, 50%, 60%, 70%, 80%, 90% or more) in the expression levels of a nucleic acid encoding sFlt-1 or an alteration (e.g., a decrease of 10%, 20%, 30%, 40%, 50%, 60%, 70%, 80%, 90% or more) in the expression levels of a nucleic acid encoding PlGF or VEGF. Methods for detecting such alterations are standard in the art and are described in Ausubel et al., supra. For example, northern blotting, Southern blotting, PCR, RNase protection assays, or real-time PCR is used to detect sFlt-1, PlGF, or VEGF mRNA levels.

In another embodiment, hybridization with PCR probes that are capable of detecting an sFlt-1 nucleic acid molecule, including genomic sequences, or closely related molecules, may be used to hybridize to a nucleic acid sequence derived from a subject determined to have or be at risk of having a fetus with trisomy 13. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations indicative of a fetus with trisomy 13 or a risk of having a fetus with trisomy 13 in an sFlt-1, PlGF, or VEGF nucleic acid molecule, or may be used to monitor expression levels of a gene encoding an sFlt-1, PlGF, or VEGF polypeptide (for example, by Northern analysis, Ausubel et al., supra).

In yet another embodiment, pregnant subjects may be diagnosed as having a fetus with trisomy 13 or a risk of having a fetus with trisomy 13 by direct analysis of the sequence of an sFlt-1, VEGF, or PlGF nucleic acid molecule.

In one embodiment, the level of sFlt-1, VEGF, or PlGF polypeptide or nucleic acid, or any combination thereof, is measured at least two different times and an alteration in the levels as compared to normal reference levels over time is used as an indicator of a fetus with trisomy 13 or a pregnant subject's risk of having a fetus with trisomy 13. In another embodiment, the level of sFlt-1, VEGF, or PlGF polypeptide or nucleic acid, or any combination thereof is compared to the level in a reference sample.

The level of sFlt-1, VEGF, or PlGF polypeptide can also be compared to a standard curve to determine if it falls within “normal ranges” of the level of polypeptide. In this embodiment, a standard curve is established for each of the polypeptides using purified or recombinant forms (e.g., greater than 80%, 90%, 95%, 99% or 100% pure) of the polypeptide for comparison. A standard curve is generated and the concentration of the polypeptide in the subject sample is determined by comparison to a standard curve established for the same polypeptide. For example, a standard curve can be established for sFlt-1 and a subject sample that, when compared to the standard curve, has sFlt-1 concentrations greater than 2 ng/mL is considered indicative of a fetus with trisomy 13 or a risk of having a fetus with trisomy 13. The standard curve described can be modified for use with PlGF or VEGF, preferably free PlGF or VEGF, as well.

The level of sFlt-1, VEGF, or PlGF in the bodily fluid of a subject having a fetus with trisomy 13 or a risk of having a fetus with trisomy 13 may be altered (increased or decreased) by as little as 10%, 20%, 30%, or 40%, or by as much as 50%, 60%, 70%, 80%, or 90% or more relative to the level of sFlt-1, VEGF, or PlGF in a normal reference.

In one embodiment, a subject sample of a bodily fluid (e.g., blood, urine, plasma, serum, amniotic fluid, or cerebrospinal fluid) is collected during pregnancy (e.g., the first, second, or third trimester), preferably early in pregnancy, for example in the first or second trimester. In another example, the sample can be a tissue or cell collected early in pregnancy, preferably in the first or second trimester. Non-limiting examples include placental tissue, placental cells, endothelial cells, and leukocytes such as monocytes. In humans, for example, maternal blood serum samples are collected from the antecubital vein of pregnant women during the first, second, or third trimesters of the pregnancy. Preferably, the assay is carried out during the first trimester, for example, at 4, 5, 6, 7, 8, 9, 10, 11, 12, or 13 weeks, or during the second trimester, for example at 14, 15, 16, 17, 18, 19, 20, 21, 22, 23, 24, 25, 26, or even 28 weeks. Such assays may also be conducted at the end of the second trimester or the third trimester, for example at 29, 30, 32, 34, 36, 37, 38, 39, or 40 weeks. It is preferable that levels of sFlt-1, VEGF, or PlGF are measured twice during pregnancy. In one example, the first measurement is in the first trimester and the second measurement is early in the second trimester. In desired embodiments, the methods of the invention are used to identify pregnant subjects at risk of having a fetus with trisomy 13. These subjects are then tested using additional genetic testing methods such as CVS, FISH, or amniocentesis for confirmation of trisomy 13. Ultrasound can also be used for further testing.

In one particular example, serial blood samples can be collected during pregnancy and the levels of soluble sFlt-1 determined by ELISA. In one study using this technique, the alternatively spliced mRNA encoding sFlt-1 is highly expressed by trophoblast cells and the protein was readily detectable in the plasma of pregnant women. It was observed that the levels of sFlt-1 increased approximately 3-fold between 20 and 36 weeks gestation. Levels were observed to be significantly higher in high-risk women who subsequently went on to develop pre-eclampsia (Charnock- Jones et al., J. Soc. Gynecol. Investig. 10(2):230, 2003).

In another example, the PAAI or sFlt-1/PlGF is determined using the serum values for each of these polypeptides. A woman identified as having a risk of having a fetus with trisomy 13 by urine analysis for PlGF can be tested further by CVS, FISH, amniocentesis, or ultrasound.

In another example, free PlGF polypeptide levels are measured in a bodily fluid sample, preferably urine, and used as a diagnostic indicator of having a fetus with trisomy 13 or a risk of having a fetus with trisomy 13. Measurements of PlGF polypeptide levels in the urine can also be used as an initial assessment of the potential risk of having a fetus with trisomy 13 and a woman determined to be “at risk” by PlGF measurements can then undergo additional diagnostic assays such as the ones described herein or known in the art. In one example, a pregnant subject diagnosed with a risk of having a fetus with trisomy 13 by free PlGF polypeptide measurement in a urine sample is further monitored by chronic villous sampling or amniocentesis. In order to detect free PlGF, it is preferred that an antibody that specifically recognizes free PlGF is used for these assays. Such an antibody can recognize, for example, the sFlt-1 binding domain of PlGF. Examples of such a specific antibody include the capture antibody used in the human PlGF ELISA kit (catalog # DPG00, R &D Systems, Minneapolis, Min.), monoclonal anti-placental growth factor (clone 37203.111, Sigma-Aldrich, St. Louis, Mo.). These antibodies recognize specific sequences in the N-terminal region of human PlGF protein. The sFlt1 binding region to PlGF is between amino acids 39-105 of the PlGF protein, wherein the total length of PlGF varies from 149 to 221 amino acids depending on the isoform of PlGF. Additional preferred antibodies include any antibody that recognizes the N-terminal region (preferably between amino acids 39-105 of PlGF) and that will specifically bind to free PlGF and not PlGF bound to sFlt-1. Antibodies raised to C-terminus will not have this property.

As with any of the diagnostic assays of the invention, PlGF levels can be compared to absolute levels known to be indicative of normal or can be compared to a reference sample or value to determine relative levels. A reference sample can be a urine sample from a subject (preferably matched for gestational age) having a known normal fetus as determined by amniocentesis or CVS. The PlGF levels can also be compared to a reference value or standard to determine absolute levels. The reference value or standard can be determined using a standard curve established based on purified or recombinant forms (e.g., greater than 80%, 90%, 95%, 99% or 100% pure) of PlGF for comparison. A value of PlGF less than 400 pg/ml, preferably less than 200 pg/ml, and most preferably less than 150 pg/ml or 100 pg/ml or a PlGF/creatinine value less than 200 pg/mg of creatinine and preferably less than 100 pg/mg of creatinine is considered a diagnostic indicator of having a fetus with trisomy 13 or a risk of having a fetus with trisomy 13. For standard curves, recombinant PlGF ranging from 10 pg/ml to 1 ng/ml can be used. Other examples of recombinant proteins that can be used to generate the standard curves include specific peptides that encompass the amino terminus of PlGF, preferably amino acids 39-105 of the PlGF protein (the region of PlGF that binds to sFlt-1). Alternatively, a recombinant PlGF NVEGF heterodimer (available commercially as catalog # 297-VP, R &D Systems, MN) can also be used. The latter has the advantage that this protein may also be used to generate the VEGF standard curve in the measurement of free VEGF.

ELISA assays are the preferred method for measuring levels of polypeptides. Desirably, antibodies are used for the ELISA but any binding agent (e.g., polypeptide, compound) that specifically binds the polypeptide to be detected can also be used. Particularly preferred, for ease and simplicity of detection, and its quantitative nature, is the sandwich or double antibody ELISA assay of which a number of variations exist, all of which are contemplated by the present invention. For example, in a typical sandwich assay, unlabeled antibody that recognizes the polypeptide (e.g., PlGF) is immobilized on a solid phase, e.g. microtiter plate, and the sample to be tested is added. After a certain period of incubation to allow formation of an antibody-antigen complex, a second antibody, labeled with a reporter molecule capable of inducing a detectable signal, is added and incubation is continued to allow sufficient time for binding with the antigen at a different site, resulting with a formation of a complex of antibody-antigen-labeled antibody. The presence of the antigen is determined by observation of a signal which may be quantitated by comparison with control samples containing known amounts of antigen.

In one example of the quantitative sandwich ELISA for free PlGF, a solid support (e.g., a microtiter plate or a membrane) is pre-coated with an anti-free PlGF binding agent (e.g., a primary antibody). Standards or samples are added to the substrate and free PlGF, if present, will bind to the antibody. A standardized preparation of enzyme-conjugated antibody that also recognizes free PlGF is then added to “sandwich” the PlGF now immobilized on the plate. The substrate is added and the enzyme and substrate are allowed to react over a short incubation period. The enzyme-substrate reaction is terminated and the change is measured by art known methods (e.g., by eye, using a spectrophotometer, or measuring chemiluminescence). Such an assay can be used to determine the relative level of free PlGF (e.g., as compared to the level in a reference sample, standard or level) or to determine the absolute concentration of free PlGF. If so desired, the concentration of free PlGF can be determined using a set of calibration standards of purified PlGF (e.g., recombinant) at varying concentrations. The calibration standards are assayed at the same time as the sample and are used to produce a standard curve measured by, for example, optical density, versus PlGF concentration. The concentration of free PlGF in the sample is then determined by comparing, for example, the optical density of the samples to the standard curve. The concentrations of free PlGF during normal pregnancy during mid-gestation and late-gestation will range from 200-800 pg/ml depending on the gestational age of the mother. Any value of urinary free PlGF less than 400 pg/ml, preferably less than 200 pg/ml or a value of urinary free PlGF less than 150 pg/mg of creatinine will be diagnostic of a pregnant subject having a fetus with trisomy 13 or at risk of having a fetus with trisomy 13. In general, the standard curves on the ELISA kit will include recombinant or purified PlGF at concentrations ranging from 10 pg/ml-1 ng/ml of PlGF.

In another example, an assay for detecting free PlGF in a urine sample includes a membrane having an immobilized PlGF binding agent that is detectably labeled in a manner that can distinguish between the PlGF binding agent when it is bound to free PlGF and when it is not bound to free PlGF. Preferred labels include fluorescent labels. The membrane is exposed to the sample for a time sufficient to allow binding of the PlGF binding agent to free PlGF present in the sample. The labeled PlGF binding agent bound to the free PlGF is then measured. Such an assay can be used to determine the relative level of free PlGF (e.g., as compared to the level from a reference sample or standard or level) or to determine the absolute concentration of free PlGF as described above. Preferred assays for the measurement of binding include fluorescence immunoassays.

In another example, an assay for detecting free PlGF in a urine sample includes a membrane having a dehydrated labeled (e.g., for colorimetric detection) PlGF binding agent (primary agent) and an immobilized anti-PlGF binding agent (secondary agent). The membrane is exposed to the sample. The sample rehydrates the labeled PlGF binding agent and if free PlGF is present in the sample, it will bind to the PlGF binding agent. The PlGF-primary agent complex will move down the membrane by capillary movement and will interact with the immobilized secondary agent. This interaction will produce a visible line from the calorimetric label at the position at which the secondary agent is immobilized.

In another example, an assay for detecting free PlGF in a urine sample includes a membrane having a dehydrated labeled (e.g., for colorimetric detection) free PlGF binding agent (primary agent), and an immobilized anti-PlGF binding agent (secondary agent). The membrane also includes purified PlGF at a threshold concentration also immobilized on the membrane. In this example, the membrane is exposed to the urine sample. The sample rehydrates the labeled primary agent and if free PlGF is present in the sample at a concentration greater than the threshold concentration, it will bind to the PlGF binding agent. The PlGF-labeled primary agent complex will move down the membrane by capillary movement. As the primary agent is already bound to the PlGF from the sample, it will not bind to the immobilized purified PlGF and no visible line will appear at this “test” position. The PlGF-primary agent complex will continue down the membrane and will interact with the immobilized secondary agent. This interaction will produce a visible line from the colorimetric label at the “control” position at which the anti-PlGF binding agent is immobilized. In this example, only one visible line will appear and will indicate a PlGF concentration above a threshold concentration. If the concentration of PlGF is below the threshold concentration, the labeled primary agent will bind to the immobilized PlGF and a visible line will appear at this “test” location as well as at the “control” location. The test assay can also include multiple test lines aimed at detecting several concentrations of PlGF in the sample. Such a graded assay is described in U.S. Pat. No. 6,660,534.

In another example, a similar membrane based assay is used but is based on standard sandwich ELISA methods. In this example, the membrane includes a reaction zone having an immobilized primary PlGF binding agent conjugated to an enzyme; a test zone having another immobilized PlGF binding agent that binds to a region of PlGF not bound by the first PlGF binding agent, and a control zone having an immobilized substance that recognizes the primary PlGF binding agent. In both the test zone and the control zone a detectable substrate for the enzyme conjugated to the first immobilized PlGF binding agent is included. The membrane is exposed to the sample and the sample moves to the reaction zone by capillary action. If PlGF is present in the sample, it binds to the first immobilized PlGF binding agent conjugated to an enzyme and forms a complex which is then carried along by capillary flow to the test zone. The PlGF-immobilized PlGF binding agent conjugated to an enzyme complex then binds to the second PlGF binding agent and forms a visible line at the location of the immobilized second PlGF binding agent (the “test” zone). The remaining first PlGF binding agent is carried along by capillary flow and will bind to the immobilized substance that recognizes or binds to the first binding agent and produce a visible line at this location (the “control” zone). If PlGF is not present in the sample, only the second line will appear at the control zone. In preferred embodiments, the first and second PlGF binding agents are antibodies and the agent that recognizes or binds to the first binding agent is a secondary anti-immunoglobulin antibody that specifically recognizes the immunoglobulin of the first antibody. The intensity of the line in the test zone can be compared to assays using a standard amount of purified PlGF protein to determine if the sample contains PlGF above or below a threshold concentration.

In any of the assays described herein, normal pregnant serum can be used as an additional control and the activity of PlGF can be measured and quantified as a percentage of PlGF activity measured from normal pregnant serum.

In preferred embodiments of any of the above-described PlGF-based diagnostic assays, the PlGF binding agent is preferably a primary antibody that recognizes free PlGF or a protein or peptide that interacts with PlGF. The secondary anti-PlGF binding agent is preferably a secondary antibody that recognizes the primary antibody or a protein that binds to the primary antibody (e.g., Protein A or Protein G), or an antibody that specifically binds the peptide that interacts with PlGF. In embodiments where the PlGF binding agent is labeled with an enzyme, the enzyme used preferably catalyzes a colorimetric reaction that can be detected by eye and/or measured by spectrophotometry. Non-limiting examples of preferred enzyme/substrate combinations are horseradish peroxidase/TMB, β-galactosidase/XGAL, and alkaline/phosphatase/1,2 dioxetane. For embodiments that include a labeled PlGF binding agent, preferred labels include colorimetric (e.g., colloidal gold), chemiluminescent, or fluorescent labels.

Any of the diagnostic ELISA assays described for PlGF above can be modified (e.g., using antibodies that specifically bind sFlt-1 or VEGF) and used for the detection of sFlt-1 or VEGF in blood serum samples.

For any of the assays described herein, the sample can be any bodily fluid. A urine sample is preferred for the PlGF-based diagnostic assays described above. The membrane can be in a standard dipstick type format or lateral flow format. The dipstick type of assay is known in the art for such assays as pregnancy detection (measuring hormone levels in that case) or urinalysis detection of creatinine or albumin in the diagnosis of kidney disease. Examples of various formats of dipstick type assays are described in U.S. Pat. No. 6,660,534, incorporated herein by reference.

In veterinary practice, assays may be carried out at any time during the pregnancy, but are, preferably, carried out early in pregnancy, preferably in the first or second trimester. Given that the term of pregnancies varies widely between species, the timing of the assay will be determined by a veterinarian, but will generally correspond to the timing of assays during a human pregnancy.

The diagnostic methods described herein can be used individually or in combination with any other diagnostic method described herein for a more accurate diagnosis of fetal trisomy 13 or a subject at risk of having a fetus with trisomy 13. In addition, the diagnostic methods described herein can be used in combination with any other diagnostic methods used to detect abnormalities during pregnancy. For example, the diagnostic methods can be combined with methods for measuring the level of any one, two, or three of alpha-feto protein (AFP), human chorionic gonadotropin (hCG), and unconjugated estriol. The measurement of all three of these proteins in maternal serum samples is commonly known as the triple screen and is usually performed at 15 to 22 weeks to detect trisomy 21 and trisomy 18 or a risk for having a fetus with trisomy 21 or trisomy 18 (see, for example, Graves et al., Am. Fam. Physician 65:915-920 (2002)). Additional screening methods used to detect abnormalities during pregnancy are known in the art and can be combined with the methods described herein (e.g., amniocentesis, CVS, FISH, ultrasound, and the screening tests described for example Lewis et al., J. Fam. Practice 53 (2004)). When used in combination with additional tests such as the triple screen, the diagnostic methods of the invention, can be performed simultaneously or within 1 day, 2, days, 5 days, 1 week, 2 weeks, 3 weeks, 5 weeks, 10 weeks, 20 weeks, up to 30 weeks within each other.

Diagnostic Kits

The invention also provides for a diagnostic test kit that includes the components required to carry out any of the diagnostic assays described above and instructions for the use of the components to diagnose fetal trisomy 13 or a subject at risk of having a fetus with trisomy 13. For example, a diagnostic test kit can include polypeptides (e.g., antibodies) or compounds that specifically bind to sFlt-1, free VEGF, or free PlGF, and components useful for detecting, and more preferably evaluating, binding between the antibodies and the sFlt-1, VEGF, or PlGF polypeptide. For detection, either the polypeptide (e.g., antibody) or compound or the sFlt-1, VEGF, or PlGF polypeptide is labeled, and either the antibody or the sFlt-1, VEGF, or PlGF polypeptide is substrate-bound, such that the sFlt-1, VEGF, or PlGF polypeptide-polypeptide (e.g., antibody) or compound interaction can be established by determining the amount of label attached to the substrate following binding between the antibody and the sFlt-1, VEGF, or PlGF polypeptide. In one example, the kit includes a free PlGF binding agent and components for detecting the presence of free PlGF. A conventional ELISA or a sandwich ELSIA is a common, art-known method for detecting antibody-substrate interaction and can be provided with the kit of the invention. sFlt-1, VEGF, or PlGF polypeptides can be detected in virtually any bodily fluid including, but not limited to urine, serum, plasma, saliva, amniotic fluid, or cerebrospinal fluid. A kit that determines an alteration in the level of sFlt-1, VEGF, or PlGF polypeptide relative to a reference, such as the level present in a normal control, is useful as a diagnostic kit in the methods of the invention. The kit can also include purified proteins (e.g., produced using recombinant methods) to be used as standards in the assay used to detect the level of sFlt-1, VEGF, or PlGF. Desirably, the kit will contain instructions for the use of the kit. In one example, the kit contains instructions for the use of the kit for the diagnosis of fetal trisomy 13 in a pregnant subject or a pregnant subject at risk of having a fetus with trisomy 13.

In one embodiment of the invention, such a kit includes a solid support (e.g., a membrane or a microtiter plate) coated with a primary agent (e.g., an antibody or protein that recognizes the antigen), standard solutions of purified protein for preparation of a standard curve, a body fluid (e.g. serum or urine) control for quality testing of the analytical run, a secondary agent (e.g., a second antibody reactive with a second epitope in the antigen to be detected or an antibody or protein that recognizes the primary antibody) conjugated to a label or an enzyme such as horse radish peroxidase or otherwise labeled, a substrate solution, a stopping solution, a washing buffer and an instruction manual.

In the examples described below, we describe our discovery that increased levels of sFlt-1 and decreased levels of PlGF are associated with fetal trisomy 13 during pregnancy. These findings support the utility of diagnostic tests that include measurement of the level of sFlt-1, PlGF, or the ratio of sFlt-1/PlGF for the diagnosis of the likelihood of fetal trisomy 13 in a pregnant subject.

### EXAMPLES

The following examples are for the purposes of illustrating the invention, and should not be construed as limiting.

### Example 1

### sFlt-1 and PlGF as Diagnostic/Predictive Indicators of Trisomy 13.

We performed a study to determine the serum concentrations of sFlt-1 and PlGF in pregnancies with chromosomal abnormalities (trisomy 13, 18 and 21) and in pregnancies with normal karyotypes. In this study, we have demonstrated that pregnant women carrying trisomy 13 fetuses, but not other trisomies, tend to have modestly higher circulating sFlt-1 and significantly lower PlGF levels. We have demonstrated that alterations in sFlt-1 and PlGF expression levels are specific for trisomy 13 pregnancies. In addition, the ratio of sFlt-1/PlGF was found to be significantly increased in trisomy 13 pregnancies as compared to other trisomies.

Maternal Serum Specimens

Archived first trimester and second trimester specimens obtained as part of an early gestation fetal malformation screening program were used for this study. Maternal serum specimens from pregnancies with trisomy 13, 18, or 21 and those with normal karyotyes were obtained from the archived serum bank of the Foundation for Blood Research (FBR), Scarborough, Me., USA. All serum specimens were collected prior to knowledge of the karyotype. A total of seventeen serum samples from trisomy 13 pregnancies were identified and each matched with 5 samples from control normal karyotype pregnancies. Control specimens were matched with each of the trisomy 13 cases for collection location, parity (primiparous vs. multiparous), maternal age (to within two years) and length of time in the freezer (within one month). Out of the seventeen trisomy 13 cases, 6 were first trimester samples, matched with 30 controls, and 11 were second trimester samples matched with 55 controls (total of 85 controls). Nine cases were primiparous patients, matched with 45 controls, while 8 were multiparous, matched with 40 controls. Additionally, samples from twenty trisomy 18 pregnancies and seventeen trisomy 21 pregnancies were included. The study was approved by the FBR Institutional Review Board.

sFlt-1 and PlGF Serum Levels

Measurement of sFlt-1 and free PlGF serum concentrations were performed by personnel who were blinded to the outcome of the pregnancy. Enzyme-linked rmmunosorbent assays (ELISA) for human sFlt-1 and free PlGF were performed in duplicate, as previously described (Levine et al., N. Engl. J. Med. 350:672-683 (2004)), with the use of commercial kits (R&D Systems, MN). The minimal detectable doses in the assays for sFlt-1 and free PlGF were 5 pg per milliliter. The short term coefficients of variation (both within and between plates) for sFlt-1 and PlGF were 18 and 8%, respectively, as determined by multiple blinded measurements of duplicate patient samples.

Statistical Analysis

The outcome variables of this study (sFlt-1, PlGF and sFlt/PlGF ratio) are presented as means ±S.E and as median with ranges (in the text) as well as box and whisker plots with medians (in the figures) (Conover, Practical Statistics (1980)). For between-group comparison, the non-parametric Mann-Whitney test was applied because the distributions of sFlt-1 and PlGF were highly skewed. All tests were two-tailed, and a p-value of 0.05 or less was considered statistically significant. SPSS software was used for analysis.

Results

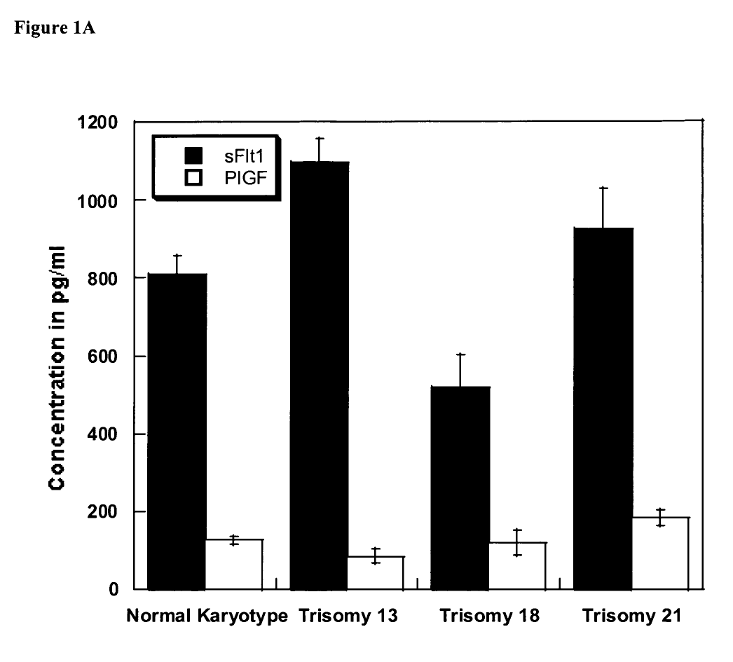

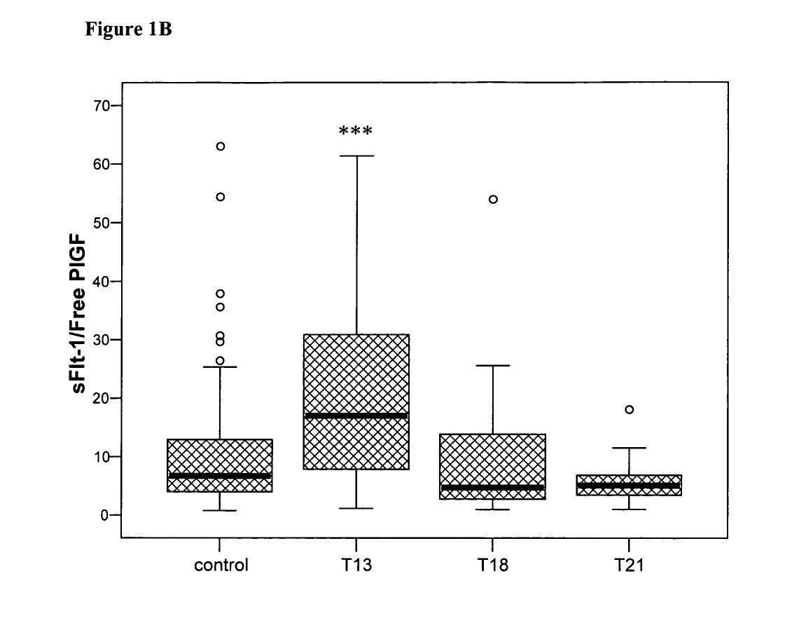

The clinical characteristics of the patients used in this study are described in the materials and methods. Serum sFlt-1 and PlGF concentrations were analyzed in all the control and trisomy specimens. The mean sFlt-1 concentrations were 810.38±46.04 pg/ml for the normal karyotype control specimens, 1096.97±160.73 pg/ml for the trisomy 13 specimens, 516.48±87.24 pg/ml for the trisomy 18 specimens and 926.18±104.06 pg/ml for the trisomy 21 specimens (See FIG. 1A). The mean PlGF levels were 126.40±9.57 pg/ml for the normal karyotype specimens, 85.75±17.42 pg/ml for the trisomy 13 specimens, 119.41±32.34 pg/ml for the trisomy 18 specimens, and 183.61±20.17 pg/ml for the trisomy 21 specimens (FIG. 1A).

The median sFlt-1 concentrations were 675.4 pg/ml (range 215.2-2331.9) for the normal karyotype control specimens, 780.5 pg/ml (range 255.2-2134.2) for the trisomy 13 specimens, 341.6 pg/ml (range 150.0-1447.6) for the trisomy 18 specimens and 919.9 pg/ml (range 223.4-1691.3) for the trisomy 21 specimens. Although the median sFlt-1 concentration in the trisomy 13 group was modestly higher than controls, the data were not statistically significant. sFlt-1 concentrations in the trisomy 18 and 21 were also not significantly altered when compared to controls. The median PlGF concentrations were 113.3 pg/ml (range 23.5-454.9) for the normal karyotype specimens, 49.5 pg/ml (range 19.3-249.6) for the trisomy 13 specimens, 56.1 pg/ml (range 21.6-570.2) for the trisomy 18 specimens, and 151.6 pg/ml (range 76.1-350.6) for the trisomy 21 specimens. The PlGF decrease in the trisomy 13 specimens was statistically significant when compared to controls (p=0.03). Trisomy 18 and 21 specimens did not have a significant decrease in PlGF concentrations when compared to controls. These data suggest that pregnant women carrying trisomy 13 fetuses but not other trisomies tend to have modestly higher circulating sFlt-1 and significantly lower PlGF.

The ratio of sFlt-1/PlGF is a more reliable index of the circulating angiogenic state than either of the markers alone and was found to be better predictor of PE risk (Levine et al., JAMA 293:77-85 (2005)). Hence, we calculated the ratios of sFlt-1/PlGF for all the specimens. The median ratios of sFlt-1/PlGF were 6.7 (range 0.8-62.9) for the normal karyotype control specimens, 17.0 (range 1.2-61.3) for the trisomy 13 specimens, 4.8 (range 0.9-53.9) for the trisomy 18 specimens and 5.1 (range 1.0-18.1) for the trisomy 21 specimens (FIG. 1B). The increased ratio observed in the trisomy 13 specimens was highly statistically significant (p=0.003) when compared to control specimens, while the sFlt-1/PlGF ratio was not significantly altered in the other trisomies.

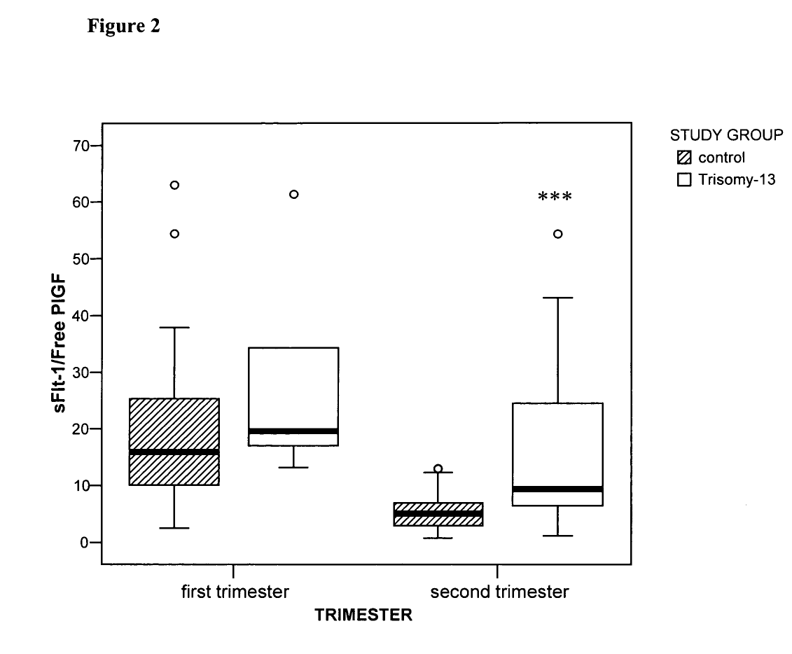

Since this study included a mixture of first trimester and second trimester specimens, we then studied the changes of sFlt-1 and PlGF in the trisomy 13 cases and the controls based on gestational ages. Although only six specimens were collected during the first trimester in the trisomy 13 group, the mean sFlt-1/P IGF ratio was nevertheless modestly elevated (by about 23%) compared to the normal karyotype controls (19.6 vs. 15.9) (FIG. 2). The ratios of sFlt-1 /PlGF were significantly elevated in the second trimester trisomy 13 cases compared with normal karyoype controls (9.4 vs. 5.1, P=0.007) (FIG. 2). These data suggest that although there were modest differences in these angiogenic factors in early gestation, they became more apparent at later gestation. These findings are consistent with the dramatic alterations in sFlt-1 and PlGF noted in pregnancies complicated by pre-eclampsia during second and early third trimester in the weeks preceding clinical symptoms.

### CONCLUSIONS

We have previously hypothesized that alterations in circulating angiogenic factors (sFlt-1, free PlGF and free VEGF) may play a pathogenic role in pre-eclampsia (Maynard, et al., J. Clin. Invest. 111:649-658 (2003)). Increased sFlt-1 and decreased free PlGF have been found in preeclamptics not only during clinical disease, but even prior to clinical symptoms (Levine et al., N. Engl. J. Med. 350:672-683 (2004); Levine et al., JAMA 293:77-85 (2005); Thadhani et al., J. Clin. Endocrinol. Metab. 89:770-775 (2004)). Women with trisomy 13 pregnancies have a higher rate of pre-eclampsia, regardless of parity (Boyd et al., Lancet 2:425-427 (1987); Boyd et al., Clin. Genet. 47:17-21 (1995)). Mothers carrying fetuses with other chromosomal abnormalities, such as trisomy 18 or trisomy 21, have rates of pre-eclampsia that are similar to the general population. Since the gene for Flt-1 and sFlt-1 is coded on chromosome 13, we hypothesized that trisomy 13 pregnancies might have a higher basal level of circulating sFlt-1 and would produce more sFlt-1 with advancing gestation as compared to normal karyotype controls. Since high circulating sFlt-1 is associated with a decreased free PlGF, we further hypothesized that free PlGF might be decreased in trisomy 13 pregnancies and the ratio of sFlt-1/PlGF would be increased. In this case control study, we have compared 17 trisomy 13 cases with 85 controls, carefully matched for maternal age, trimester of pregnancy, parity and freezer storage time (5 controls for each case). Additional control was provided by looking at two other trisomies, 18 and 21. We have now demonstrated that circulating sFlt-1 was on average 35% higher and circulating free PlGF 32% lower in trisomy 13 pregnancies during early gestation, as compared to normal karyotype controls. We have also demonstrated that the ratio of sFlt-1/PlGF was substantially altered in trisomy 13 cases as compared to normal karyotype controls and that this difference is more apparent during second trimester than during first trimester. Furthermore, the ratio of sFlt-1/PlGF was not elevated in other trisomies such as 18 or 21. Finally, the alterations in sFlt-1 and PlGF observed in trisomy 13 patients during first and second trimester were similar to the changes previously reported in pregnancies that subsequently developed pre-eclampsia (Levine et al., N. Engl. J Med. 350:672-683 (2004); Levine et al., JAMA 293:77-85 (2005); and Thadhani et al., supra)). These data indicate that the increased sFlt-1 and decreased free PlGF noted in trisomy 13 patients may contribute to increased risk of pre-eclampsia previously reported in these patients. We have previously hypothesized that the abnormal placentation of pre-eclampsia leads to the elaboration of anti-angiogenic factors which in turn induces the hypertensive syndrome (Maynard et al., J. Clin. Invest. 111:649-58 (2003); Levine et al., (2004) and (2005), supra). An interesting result is that PlGF was significantly higher in the trisomy 21 samples than in the controls, consistent with some of the published literature (Spencer et al., Prenat. Diagn. 21:718-722 (2001); Su et al., Prenat. Diagn. 22:8-12 (2002)), but contradicting other references (Debieve et al., Mol. Hum. Reprod. 7:765-770 (2001); Lambert-Messerlian et al., Prenat. Diagn. 24:876-880 (2004)). The average trisomy 21 ratio sFlt-1/PlGF was lower than the equivalent control value, although did not reach significance (P=0.09).

Certain pregnancy complications that have excess trophoblastic tissue such as multifetal pregnancy, hydatidiform mole, and triploidy have also been long known to predispose to pre-eclampsia. The fact that pregnancies with excess fetal genetic material are more susceptible to pre-eclampsia may emphasize the fetal genomic contribution to pre-eclampsia. Previous data suggesting a possible link between pregnancies with excess fetal genetic material and pre-eclampsia led to the hypothesis that gene products encoded on chromosome 13 may play pathogenic role in the development of pre-eclampsia. Several candidate factors encoded by chromosome 13 such as clotting factors X and VII, and collagen type 4 were hypothesized to play a role in pre-eclampsia; however none has been shown to be important (Tuohy et al, Br J Obstet Gynaecol 1992, 99:891-4).

Our identification of the relevance of the Flt-1/sFtl-1 locus on chromosome 13, and our discovery of the linkage between trisomy 13 and a higher maternal serum level of sFlt-1 and decreased serum free PlGF provides a plausible explanation for the association between pregnancies with excess fetal genetic material and a propensity towards pre-eclampsia.

In summary, these results suggest that trisomy 13 pregnancies have increased circulating sFlt-1 and decreased free PlGF as compared to trisomy 18 or trisomy 21 or normal karyotype pregnancies. Since we have previously observed similar alterations in circulating angiogenic proteins in pregnancies that are subsequently complicated by pre-eclampsia, it is likely that the increased risk of pre-eclampsia noted in trisomy 13 patients may be directly related to the alterations in the angiogenic profile.

### Other Embodiments

From the foregoing description, it is apparent that variations and modifications may be made to the invention described herein to adopt it to various usages and conditions. Such embodiments are also within the scope of the following claims.

All patents, patent applications, and publications mentioned in this specification are herein incorporated by reference to the same extent as if each independent patent, patent application, or publication was specifically and individually indicated to be incorporated by reference. In addition, U.S. application publication Nos. 20040126828, 20050025762, and 20050170444 and PCT patent application publication numbers WO2004/008946A2 and WO 2005/077007 are hereby incorporated by reference in their entirety.

What is claimed is:

1. A method of diagnosing a pregnant subject as having, or at risk for having, a fetus with trisomy 13, said method comprising measuring the level of at least one of sFlt-1, VEGF, or PlGF polypeptide in a sample from said pregnant subject.

2. The method of claim 1, wherein said sample is serum and a level of sFlt-1 greater than 2 ng/ml serum diagnoses said subject as having, or at risk of having, a fetus with trisomy 13.

3. The method of claim 1, wherein said sample is a serum sample and a level of free PlGF less than 200 pg/ml serum diagnoses said subject as having, or at risk of having, a fetus with trisomy 13.

4. The method of claim 1, further comprising comparing the level of said at least one of sFlt-1, VEGF or PlGF polypeptide to a reference, wherein an alteration in said levels in said pregnant subject sample relative to said reference diagnoses said subject as having, or at risk of having, a fetus with trisomy 13.

5. The method of claim 4, wherein said reference is a prior sample taken from said subject.

6. The method of claim 4, wherein said reference is a normal reference sample or value.

7. The method of claim 4, wherein a decrease in said sFlt-1 levels or an increase in said VEGF or PlGF level as compared to said reference diagnoses said subject as having, or at risk of having, a fetus with trisomy 13.

8. The method of claim 1, wherein said VEGF or PlGF is free VEGF or free PlGF.

9. The method of claim 1, comprising measuring the levels of at least two of sFlt-1, VEGF, and PlGF polypeptides in a sample from said subject and calculating the relationship between said levels of sFlt-1, VEGF, or PlGF using a metric.

10. The method of claim 9, wherein said metric is a pre-eclampsia anti-angiogenic index (PAAI):[sFlt-1/VEGF+PlGF].

11. The method of claim 10, wherein a PAAI value greater than 20 diagnoses said subject as having, or at risk for having, a fetus with trisomy 13.

12. The method of claim 9, wherein said metric is sFlt-1/PlGF.

13. The method of claim 12, wherein a sFlt-1/PlGF ratio value greater than 15 diagnoses said subject as having, or at risk for having, a fetus with trisomy 13.

14. The method of claim 9, further comprising measuring the levels of at least two of sFlt-1, VEGF, and PlGF polypeptides in a reference sample and calculating the relationship between said levels of sFlt-1, VEGF, or PlGF using a metric and comparing the metric value from said subject sample with the metric value from said reference sample, wherein an alteration in the metric value, diagnoses said subject as having, or at risk for having, a fetus with trisomy 13.

15. The method of claim 14, wherein said metric is PAAI and an increase in said PAAI in said subject sample as compared to said reference sample diagnoses said subject as having, or at risk for having, a fetus with trisomy 13.

16. The method of claim 14, wherein said metric is sFlt-1/PlGF, and an increase in said sflt-1/PlGF in said subject sample as compared to said sFlt-1/PlGF in said reference sample diagnoses said subject as having, or at risk for having, a fetus with trisomy 13.

17. The method of claim 1, wherein said measuring is done using an immunological assay.

18. The method of claim 1, wherein said sample is a bodily fluid, a cell, or a tissue of said subject.

19. The method of claim 18, wherein said bodily fluid is selected from the group consisting of urine, amniotic fluid, serum, plasma, or cerebrospinal fluid.

20. The method of claim 18, wherein said cell is selected from the group consisting of: an endothelial cell, a leukocyte, a monocyte, and a cell derived from the placenta.

21. The method of claim 18, wherein said tissue is a placental tissue.

22. The method of claim 1, wherein said subject is a human.

23. The method of claim 1, wherein said subject is a non-human selected from the group consisting of: a cow, a horse, a sheep, a pig, a goat, a dog, or a cat.

24. The method of claim 1, wherein at least one of said levels measured is the level of sFlt-1.

25. The method of claim 24, wherein when the level of sFlt-1 is measured then the level of PlGF is also measured.

26. The method of claim 1, wherein said measuring of levels is done on two or more occasions and an alteration in said levels between measurements diagnoses said subject as having, or at risk of having, a fetus with trisomy 13.

27. The method of claim 26, wherein the first of said two or more occasions is during the first trimester and the second of said two or more occasions is during the second trimester.

28. The method of claim 1, further comprising measuring the level of at least one protein selected from the group consisting of: alpha-feto protein, human chorionic gonadotropin, and unconjugated estriol in said sample from said pregnant subject.

29. A method of diagnosing a subject as having, or at risk for having, a fetus with trisomy 13, said method comprising measuring the level of free PlGF in a urine sample from said subject.

30. The method of claim 29, wherein a level of free PlGF in said urine sample less then 400 pg/ml urine measured during the second or third trimester diagnoses said subject as having, or at risk of having, a fetus with trisomy 13.

31. The method of claim 29, further comprising measuring the level of creatinine in said urine sample, wherein a level of free PlGF less than 200 pg per mg of creatinine in said sample diagnoses said subject as having, or at risk of having, a fetus with trisomy 13.

32. The method of claim 29, further comprising comparing said level of free PlGF from said subject to the level of PlGF from a reference sample, wherein a decrease in said free PlGF from said subject compared to said reference sample diagnoses said subject as having, or at risk of having, a fetus with trisomy 13.

33. The method of claim 32, wherein said reference sample is a prior sample taken from said subject.

34. The method of claim 33, wherein the subject sample is taken during the second trimester and the reference sample is taken during the first trimester.

35. The method of claim 29, further comprising measuring the level of at least one of sFlt-1, PlGF, and VEGF polypeptide in a sample from said subject, wherein said sample is a bodily fluid selected from the group consisting of urine, blood, amniotic fluid, serum, plasma, or cerebrospinal fluid.

36. The method of claim 35, wherein the level of sFlt-1 from a sample of serum from said subject is measured.

37. The method of claim 35, wherein the level of sFlt-1 and PlGF from a sample of serum from said subject is measured.

38. The method of claim 35, further comprising comparing said level of sFlt-1, PlGF, or VEGF polypeptide from said subject to the level of sFlt-1, PlGF, or VEGF polypeptide in a reference sample.

39. The method of claim 38, wherein an increase in said level of sFlt-1 or a decrease in said level of VEGF or PlGF polypeptide from said subject sample compared to said reference sample diagnoses said subject as having, or at risk of having, a fetus with trisomy 13.

40. The method of claim 35, comprising measuring the level of at least two of said sFlt-1, VEGF, and PlGF polypeptides, and calculating the relationship between said levels of at least one of sFlt-1, VEGF, and PlGF using a metric.

41. The method of claim 40, further comprising measuring the level of at least two of sFlt-1, VEGF, and PlGF from a reference sample and calculating the relationship between said levels using a metric, and comparing the metric from said subject sample to the metric from said reference sample, wherein an alteration in said metric diagnoses said subject as having, or at risk of having, a fetus with trisomy 13.

42. The method of claim 41, wherein said metric is sFlt-1/PlGF or PAAI.

43. The method of claim 42, wherein an increase in the sFlt-1/PlGF or PAAI value from said subject sample relative to said reference sample diagnoses said subject as having, or at risk of having, a fetus with trisomy 13.

44. A kit for the diagnosis of fetal trisomy 13, or a risk of, having a fetus with trisomy 13, in a pregnant subject, comprising a binding agent that specifically binds a sFlt-1, VEGF, or PlGF polypeptide, or any combination thereof, and instructions for the use of said kit for the diagnosis of fetal trisomy 13, or a risk of having a fetus with trisomy 13 in a pregnant subject.

45. The kit of claim 44, wherein said binding agent is an antibody that specifically binds sFlt-1, VEGF, or free PlGF.

46. The kit of claim 44, wherein said kit comprises a free PlGF binding agent for detecting free PlGF polypeptide.

47. The kit of claim 46, wherein said free PlGF binding agent is immobilized on a membrane.

48. The kit of claim 47, wherein said membrane is supported on a dipstick structure and the sample is deposited on the membrane by placing the dipstick structure into the sample.

49. The kit of claim 44, further comprising a reference sample or value.

50. The kit of claim 44, further comprising a component for measuring the level of at least one protein selected from the group consisting of: alpha-feto protein, human chorionic gonadotropin, and unconjugated estriol.
